(19) United States
(12) Patent Application Publication
(10) Pub. No.: US 2026/0076565 A1
(43) Pub. Date: Mar. 19, 2026

(54) SYSTEMS AND METHODS FOR CONDUCTING OCCUPATIONAL VISION TESTING USING SIMULATED UNDERWATER ENVIRONMENTS

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/885,449

(22) Filed: Sep. 13, 2024

(51) Int. Cl. A61B 3/113; A61B 3/00

(52) U.S. Cl. CPC A61B 3/113; A61B 3/005

(57) ABSTRACT

A user's visual capabilities can be evaluated via a virtual reality (VR) system, which can include a VR headset in electronic communication with a computing device. The computing device causes simulated underwater environments, which can include objects, optotypes, and various lighting conditions, to be displayed on the VR headset. Using varying combinations of eye-tracking sensors, eye-tracking cameras, motion-tracking sensors, handheld devices, and microphones, the VR headset collects data about the user as she participates in visual tasks within the simulated underwater environments. Optionally, advanced algorithms in the computing device dynamically alter the simulated underwater environments and the visual tasks and analyze the user's responses to evaluate the user's underwater visual capabilities and her qualification for jobs that require her to work underwater.

### BACKGROUND

### Field of the Inventions

The present application relates to methods of assessing various ocular conditions through extended reality systems. More specifically, methods and systems are applied to conduct visual tasks and exams in extended reality environments to evaluate a user's underwater visual capabilities and perform training for underwater occupational environments.

### Description of the Related Art

As virtual reality (VR) technology has become increasingly sophisticated, new highly immersive experiences have been made possible through improvements in head and motion tracking systems. Eye-tracking technology allows systems to detect and respond to where the user is looking. This capability enhances user interaction and makes virtual environments more responsive and engaging. Eye tracking is being integrated into a variety of VR applications, from gaming and training simulations to medical diagnostics and research, as it offers a more intuitive way for users to interact with digital content.

### SUMMARY

The systems, methods and devices of this disclosure each have several innovative aspects, no single one of which is solely responsible for the desirable attributes disclosed herein.

Despite the advancements in VR technology, and in particular, eye-tracking technology, in accordance with some embodiments disclosed herein is the realization that VR technology can provide unique eyecare solutions through monitoring and tracking one or more of the user's eyes and providing a diagnostic, treatment protocol, and/or treatment system. Indeed, in accordance with some embodiments disclosed herein is the realization that VR technology can be used to address challenges associated with diagnosing a variety of eye disorders and ocular conditions, such as detecting misalignment, macular degeneration, tear film characteristics, floater characteristics, eye tracking issues, motion sensitivity, and other eye movement disorders and the treatment of such.

Some embodiments of this disclosure include a VR system that can test and train vision adaptability by simulating real-world scenarios. This VR system can include a high-resolution VR headset integrated with precision eye-tracking technology and specialized software capable of generating immersive and interactive environments that replicate a wide range of real-world conditions. Users may wear the VR headset and are led through simulations of everyday activities, such as reading, driving, navigating through busy streets, and performing sports activities. The eye-tracking sensors can monitor the user's gaze direction, fixation stability, and visual response accuracy, while the software can dynamically adjust the scenarios to assess and improve the user's ability to adapt to different visual challenges, such as changes in lighting, movement, and focus distances.

Optionally, the VR application can include a variety of training modules designed to enhance visual adaptability. These modules can feature tasks that require users to quickly shift their focus between near and far objects, track moving targets, and adjust to varying light conditions. The software can process the data in real-time using advanced algorithms to evaluate parameters such as reaction time, visual acuity, and the ability to maintain focus and clarity under changing conditions. Based on the analysis, the system can provide personalized feedback and adaptive training exercises to improve the user's visual adaptability. The results can be compiled into a comprehensive report that offers detailed insights into the user's visual performance, identifying strengths and areas for improvement. This method offers a dynamic, engaging, and precise approach to testing and training vision adaptability, representing a significant advancement over traditional static vision tests.

The VR-based vision adaptability training and testing system can utilize a high-quality VR headset, such as the Oculus Quest 2, which may be integrated with high-precision eye-tracking technology. The eye-tracking sensors can include infrared cameras capable of capturing detailed eye movements, fixation points, and response times with high accuracy and minimal latency. The software development can involve creating a library of interactive real-world scenarios designed to test and enhance various aspects of visual adaptability. These scenarios ca include tasks that prompt users to quickly adjust their focus between objects at different distances, track moving targets, and adapt to changes in lighting and motion.

The system can be calibrated using a control group of individuals with diverse visual profiles to establish baseline performance metrics and validate the accuracy of the adaptability assessment algorithms. Users can then operate the system by wearing the VR headset and participating in the guided training and testing modules within the virtual environments. Based on the user's performance, the system can generate a detailed report outlining their vision adaptability, providing personalized feedback and recommending targeted training exercises to improve visual performance. This approach offers a precise, non-invasive, and user-friendly method for testing and training vision adaptability, providing substantial benefits for both clinical applications and personal eye care routines.

Another embodiment of this disclosure can include a VR method for creating personalized vision therapy sessions tailored to address user-specific visual deficiencies. This approach offers a precise, non-invasive, and user-friendly method for delivering personalized vision therapy, providing substantial benefits for both clinical applications and personal eye care routines.

Optionally, this system can comprise a high-resolution VR headset integrated with precision eye-tracking technology and specialized software capable of generating customized visual therapy exercises within immersive virtual environments. The eye-tracking sensors can include infrared cameras capable of capturing detailed eye movements, fixation patterns, and response times with high accuracy and minimal latency. The software development involves creating a comprehensive library of vision therapy exercises designed to address various visual deficiencies. The VR headset is worn by users and displays an initial assessment, which can be made up of vision tests directed towards identifying specific visual deficiencies (e.g., strabismus, amblyopia, or convergence insufficiency). The system compiles the user's performance data into detailed progress reports, which can be reviewed by eye care professionals to ensure the therapy is effective and to make any necessary adjustments.

Optionally, the software can generate a personalized vision therapy plan that can include a variety of interactive exercises designed to improve the identified visual deficiencies. These exercises may involve tasks such as tracking moving objects, focusing on targets at varying distances, and performing coordination drills that challenge the user's visual system in targeted ways. The VR application can also adjust the difficulty level of the exercises in real-time, providing adaptive feedback and progressively increasing the challenge as the user's visual performance improves. The system can record and analyze the user's performance data, offering insights and progress reports that can be used by eye care professionals to further refine and optimize the therapy sessions. This method offers a dynamic, engaging, and highly effective approach to vision therapy, representing a significant advancement over traditional, static therapy methods.

Optionally, the VR system can undergo calibration using a control group of individuals with known visual deficiencies to establish baseline performance metrics and validate the effectiveness of the therapy algorithms.

In another embodiment of this disclosure, a VR software teaches and tests eye relaxation techniques within stress-induced visual environments. This method offers a precise, dynamic, engaging, non-invasive, and user-friendly approach to teaching and testing eye relaxation techniques, representing a significant advancement over traditional static exercises.

This system can comprise a high-resolution VR headset integrated with precision eye-tracking technology and specialized software capable of generating immersive environments that simulate visually stressful conditions, such as prolonged screen exposure, glare, and high-contrast settings. The eye-tracking sensors can include infrared cameras capable of capturing detailed eye movements, blink rates, and fixation stability with high accuracy and minimal latency. The software can provide real-time feedback and adjust the difficulty of the scenarios based on the user's responses. The software can also process the data in real-time, using advanced algorithms to evaluate the effectiveness of the relaxation techniques in reducing visual stress indicators, such as blink rate and gaze stability. The results can be compiled into a comprehensive report that can provide detailed insights into the user's ability to manage visual stress, recommend specific relaxation techniques for the user's daily routine, and highlight improvements in visual stress management.

Optionally, the VR software can include interactive modules where users learn and practice relaxation techniques in progressively challenging visual environments. For instance, users may start with basic exercises in a low-stress setting and gradually move to more demanding scenarios, such as simulating an 8-hour workday on a computer or navigating through environments with intense lighting. Users wear the VR headset and are taught various eye relaxation techniques, such as the 20-20-20 rule, palming, and guided eye movements.

Optionally, the software development involves creating a comprehensive library of interactive modules designed to teach and test various eye relaxation techniques. These modules should simulate different visually stressful environments, allowing users to practice relaxation techniques in a controlled yet challenging setting. Moreover, the system can be calibrated using a control group of individuals with known visual stress profiles to establish baseline performance metrics and validate the effectiveness of the relaxation training algorithms.

Another embodiment of the present disclosure is a VR system that simulates underwater vision scenarios for specialized occupational vision testing. This system offers a non-invasive, user-friendly, dynamic, engaging, and precise approach to underwater vision testing, representing a significant advancement over traditional methods that rely on simulated pools or static tests and providing substantial benefits for specialized occupational training and safety.

The VR system can include a high-resolution VR headset integrated with precision eye-tracking technology and specialized software capable of generating realistic underwater environments. The eye-tracking sensors can monitor the user's gaze direction, fixation stability, and response accuracy, while the software can dynamically adjust the visual scenarios to assess and train the user's visual performance in underwater settings.

The software can process the data in real-time using advanced algorithms to evaluate parameters such as visual clarity, depth perception, and ability to adapt to changes in water conditions. The results can be compiled into a comprehensive report that can provide detailed insights into the user's underwater visual performance, identify strengths and areas for improvement, and recommend steps for vision improvement occupational training. The software development involves creating a library of underwater scenarios designed to test various aspects of visual performance in water. Once the hardware and software components are integrated, the system undergoes calibration using a control group of individuals with known underwater vision profiles to establish baseline performance metrics and validate the accuracy of the testing algorithms.

Optionally, when users wear the VR headset, they can be shown a series of tasks that replicate the visual conditions encountered underwater (e.g., varying water clarity, light refraction, and movement through water). This approach is particularly useful for occupations such as divers, underwater welders, and marine biologists, where accurate vision is critical for safety and effectiveness.

Optionally, the VR application can include a variety of interactive scenarios that challenge different aspects of underwater vision, such as identifying objects at different distances and depths, navigating through underwater environments, and performing precision tasks under simulated water conditions.

Another embodiment of the present disclosure is a VR method that simulates outdoor scenarios to test how the user's vision adapts to different weather conditions. This approach is particularly beneficial for occupations that require precise vision in varying outdoor conditions, such as pilots, drivers, athletes, and outdoor workers. This method offers a non-invasive, user-friendly, dynamic, engaging, and precise approach to testing vision adaptation to weather conditions, representing a significant advancement over traditional methods that rely on static tests or limited environmental simulations and providing substantial benefits for both professional and personal applications.

Optionally, this system can comprise a high-resolution VR headset integrated with precision eye-tracking technology and specialized software capable of generating realistic outdoor environments that mimic diverse weather conditions. The eye-tracking sensors can monitor the user's gaze direction, fixation stability, and visual response accuracy. Moreover, the eye-tracking sensors can include infrared cameras capable of capturing detailed eye movements, fixation points, and response times with high accuracy and minimal latency. The software can dynamically adjust the visual scenarios to assess the user's ability to adapt to changing weather conditions. Furthermore, the software can process the data in real-time using advanced algorithms to evaluate parameters such as visual clarity, reaction time, and the ability to maintain visual performance under varying weather conditions. The results are compiled into a comprehensive report that can provide detailed insights into the user's vision adaptation capabilities under different weather conditions, identify strengths and areas for improvement, and recommend strategies for enhancing visual performance in outdoor environments.

Optionally, the software development can involve creating a library of outdoor scenarios designed to test visual performance under different weather conditions. Additionally, the system can be calibrated using a control group of individuals with varying visual profiles to establish baseline performance metrics and validate the accuracy of the adaptation assessment algorithms.

The VR system can guide users through a series of tasks that replicate real-world outdoor activities under various weather conditions, such as bright sunlight, fog, rain, snow, and low-light environments. Moreover, the VR software can include a variety of interactive scenarios that challenge different aspects of vision adaptation, such as identifying objects at different distances and under different weather conditions, navigating through complex environments with reduced visibility, and performing precision tasks in challenging weather scenarios.

The next embodiment of this application is a VR system that can simulate scenarios that require multifactorial responses from people (e.g., driving or sports) to assess and improve vision and reflexes. Using this VR system, athletes can train in a controlled yet challenging environment that provides them with valuable feedback that can be used to optimize their visual performance and reaction times in real-world sports situations. This method offers a dynamic, engaging, non-invasive, user-friendly, and precise approach to sports vision training, representing a significant advancement over traditional static vision tests and drills and providing substantial benefits for athletes and sports enthusiasts seeking to optimize their performance.

The VR vision and reflex training system can incorporate a high-quality VR headset, such as the Oculus Quest 2, with high-precision eye-tracking technology. The eye-tracking sensors monitor the user's gaze direction, fixation stability, and response times. The eye-tracking sensors should include infrared cameras capable of capturing detailed eye movements, fixation points, and response times with high accuracy and minimal latency.

The VR system can also incorporate specialized software capable of generating realistic, immersive sports environments. The software can dynamically adjust the visual scenarios to evaluate and enhance the user's sports vision and reflexes. Moreover, the software processes the data in real-time using advanced algorithms to evaluate parameters such as visual acuity, depth perception, peripheral awareness, and reaction speed. Based on the analysis, the system can generate personalized training exercises that target specific visual skills needed for different sports. Moreover, the system can generate a detailed report based on the user's performance. This report can outline the user's sports vision and reflex capabilities and recommend training exercises for improving her visual performance in specific sports. Additionally, the software development can involve creating a library of interactive scenarios designed to test and enhance various aspects of vision and reflexes.

Optionally, the system can undergo calibration using a control group of athletes with known sports vision profiles to establish baseline performance metrics and validate the accuracy of the training algorithms.

The VR system can guide users through a series of tasks that replicate various sports (e.g., baseball, soccer, tennis, and basketball), driving scenarios, and high-intensity workplaces. The VR application can include a variety of interactive scenarios designed to test different aspects of vision and reflexes. The tasks can involve tracking moving balls, predicting trajectories, quickly shifting focus between near and far objects, and making split-second decisions based on visual cues.

In other embodiments of this disclosure, a VR system can simulate occupational hazards for the purpose of evaluating and enhancing vision safety protocols across various industries. Based on the evaluation, the system can provide detailed insights the adequacy of current safety protocols. The results can be compiled into a comprehensive report that offers recommendations for enhancing vision safety measures, such as the design of protective eyewear, the implementation of better lighting conditions, and the adoption of more effective safety training programs. This method offers a dynamic, engaging, non-invasive, user-friendly, and precise approach to evaluating and improving vision safety protocols, representing a significant advancement over traditional safety assessments and providing substantial benefits for workplace safety and occupational health.

This system can comprise a high-resolution VR headset integrated with precision eye-tracking technology. The eye-tracking sensors can monitor the user's gaze direction, fixation stability, and reaction times. Moreover, the eye-tracking sensors can include infrared cameras capable of capturing detailed eye movements, fixation patterns, and response times with high accuracy and minimal latency. The software can analyze the information collected by the eye-tracking sensors and generate a detailed report outlining the effectiveness of current vision safety protocols and recommending improvements tailored to specific occupational settings.

Optionally, the VR system can include specialized software capable of generating immersive virtual environments that replicate workplace settings and potential hazards specific to industries such as construction, manufacturing, healthcare, and transportation. The software can dynamically adjust the scenarios to assess the effectiveness of existing vision safety protocols and identify areas for improvement. Moreover, the software can process the data in real-time using advanced algorithms to evaluate parameters such as visual acuity, response accuracy, and the effectiveness of eye protection measures. The software development can involve creating a library of occupational hazard scenarios designed to test vision safety protocols in various industries.

Optionally, the system undergoes calibration using a control group of workers from different industries to establish baseline performance metrics and validate the accuracy of the safety assessment algorithms.

In some embodiments, the VR system can guide users through tasks that simulate real-world job functions and expose them to potential visual hazards, such as exposure to bright lights, flying debris, and high-speed machinery. This is possible because the VR software can advantageously include a variety of interactive scenarios tailored to the specific needs and hazards of different industries. Tasks involve navigating through a construction site with moving equipment, performing precision tasks in a manufacturing plant with variable lighting, and responding to emergency situations in healthcare settings.

The applicant's related disclosures and discussion of many features relevant to aspects of the present disclosure are found in the following applications, each of which are incorporated by reference herein, in their entirety: U.S. application Ser. No. 18/811,673, filed on Aug. 21, 2024; U.S. application Ser. No. 18/811,677, filed on Aug. 21, 2024; U.S. application Ser. No. 18/811,683, filed on Aug. 21, 2024; U.S. application Ser. No. 18/811,686, filed on Aug. 21, 2024; U.S. application Ser. No. 18/811,690, filed on Aug. 21, 2024; U.S. application Ser. No. 18/811,694, filed on Aug. 21, 2024; U.S. application Ser. No. 18/811,695, filed on Aug. 21, 2024; U.S. application Ser. No. 18/811,698, filed on Aug. 21, 2024; U.S. application Ser. No. 18/811,701, filed on Aug. 21, 2024; U.S. application Ser. No. 18/811,704, filed on Aug. 21, 2024; U.S. application Ser. No. 18/811,713, filed on Aug. 21, 2024; U.S. application Ser. No. 18/811,715, filed on Aug. 21, 2024; U.S. application Ser. No. 18/811,720, filed on Aug. 21, 2024; U.S. application Ser. No. 18/811,724, filed on Aug. 21, 2024; U.S. application Ser. No. 18/811,729, filed on Aug. 21, 2024; and U.S. application Ser. No. 18/811,730, filed on Aug. 21, 2024.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is an example data processing environment having one or more servers communicatively coupled to one or more computer devices, in accordance with some embodiments.

FIG. 2 is an environment in which a computer device is applied to facilitate visual assessment or eyewear fitting, in accordance with some embodiments.

FIG. 3 is a block diagram of a computer system configured to implement vision assessment or eyewear fitting, in accordance with some embodiments.

FIG. 4 is a block diagram of a machine learning system for training and applying machine learning models, in accordance with some embodiments.

FIG. 5A is a structural diagram of an example neural network applied to process input data in a machine learning model, in accordance with some embodiments.

FIG. 5B is an example node in the neural network, in accordance with some embodiments.

FIG. 6A is an example “tumbling E” chart applied in a visual acuity test, in accordance with some embodiments.

FIGS. 6B, 6C, 6D, and 6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D can include four diagrams of example graphical user interfaces rendered to determine a visual acuity score in a virtual environment created by a headset device, in accordance with some embodiments.

FIGS. 9A-9C can include three diagrams of example graphical user interfaces rendered to determine a nearsighted or farsighted power in a virtual environment created by a headset device, in accordance with some embodiments.

FIGS. 10A-10F can include six diagrams of example graphical user interfaces rendered to determine eye stigmatism in a virtual environment created by a headset device, in accordance with some embodiments.

FIG. 11 illustrates a placement of motion-tracking sensors on a VR headset, in accordance with some embodiments.

FIGS. 12A and 12B illustrate virtual environments that simulate real-world scenarios and include real-world obscurities such as glare and rain, in accordance with some embodiments.

FIG. 13A illustrates charts of the cardinal gaze positions labeled with the extraocular muscles that correspond with the gaze positions superimposed over the user's eyes, in accordance with some embodiments.

FIG. 13B illustrates a normal pupil, a constricted pupil, and a dilated pupil, as perceived by a VR headset, in accordance with some embodiments.

FIG. 14A illustrates a virtual environment of a forest with a bug displayed in two different positions, in accordance with some embodiments.

FIG. 14B illustrates a virtual environment of a busy street with various objects at different distances from the user, in accordance with some embodiments.

FIGS. 15A and 15B illustrate a baseball flying towards a user in a VR environment, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description can include specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

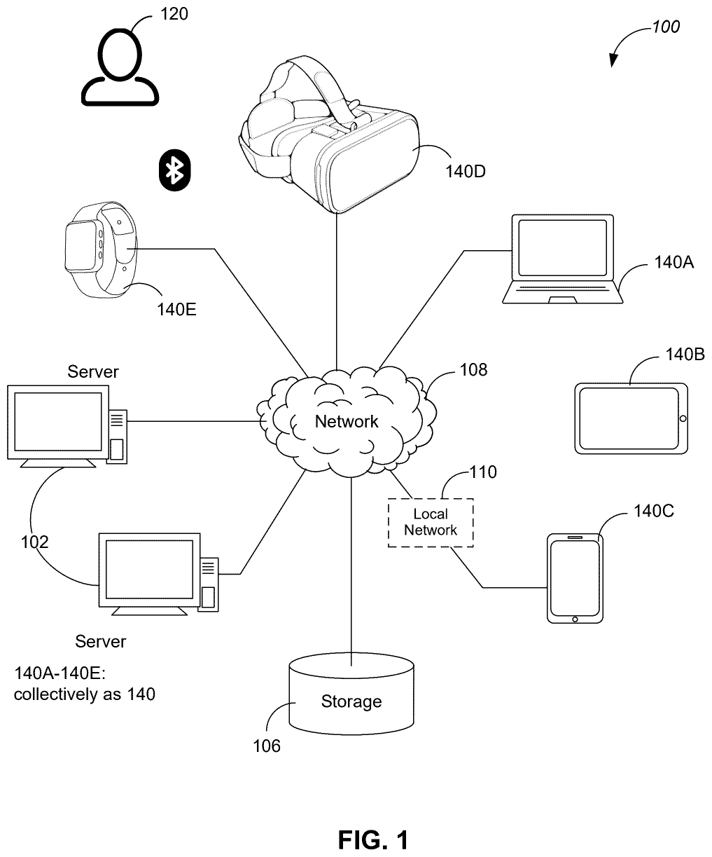

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D (also called a head-mounted display (HMD) device 140D) configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real-time data communication with the computer devices 140 that can be remote from each other or from the one or more servers 102. Further, in some embodiments, the one or more servers 102 can implement data processing tasks that are not completed locally by the computer devices 140.

For example, the computer devices 140 can include a game console (e.g., the headset device 140D) that executes an interactive online gaming application (e.g., for visual assessment or eyewear fitting). The game console can receive a user instruction and sends it to a server 102 with user data. The server 102 generates a stream of video data based on the user instruction and user data and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 can include connections, such as wire, wireless communication links, or fiber optic cables. Examples of the one or more communication networks 108 can include local area networks (LAN), wide area networks (WAN) such as the Internet, or a combination thereof. The one or more communication networks 108 are, optionally, implemented using any known network protocol can include various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D can include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound can include user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern.

For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

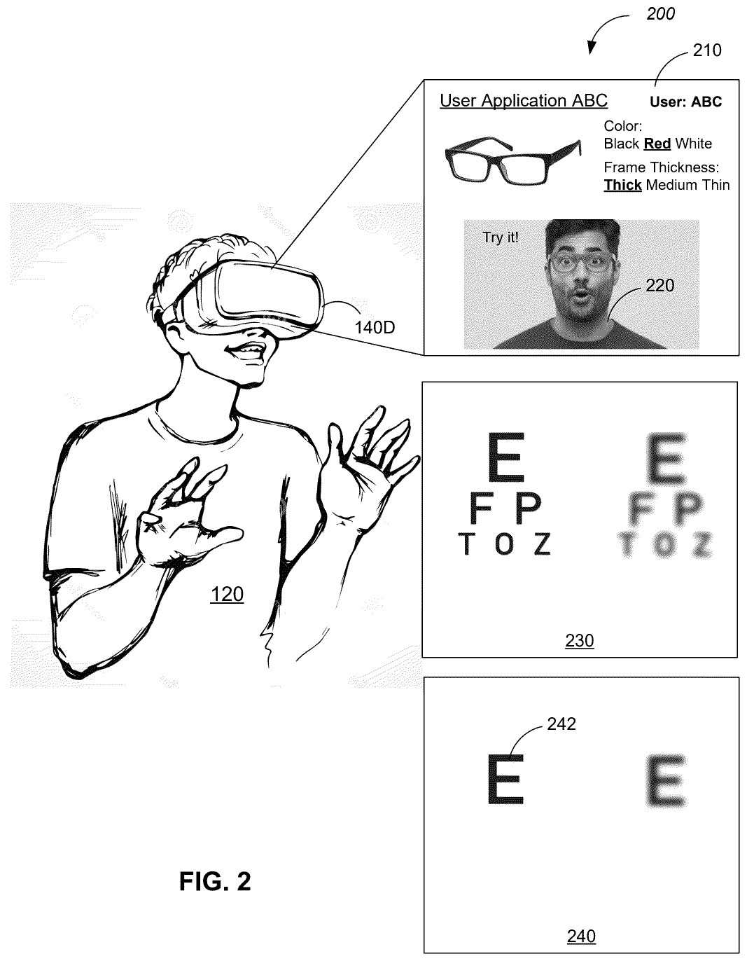

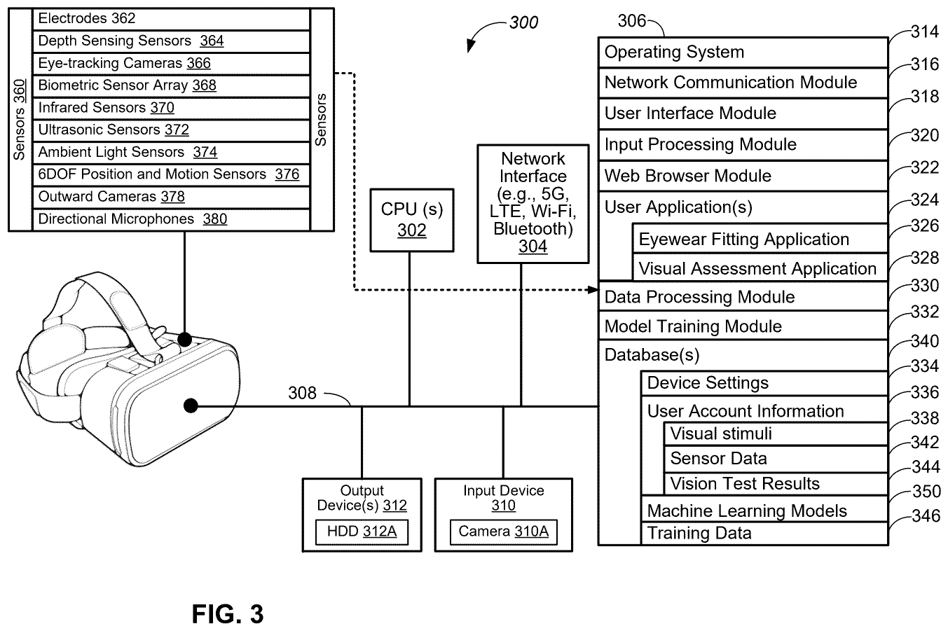

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D can include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound can include user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive, from the first user interface 210, a user feedback message indicating an issue, requesting further improvement, or confirming a fit. In some embodiments, a second user interface 230 can be displayed on a computer device 140 associated with the user 120. The second user interface 230 can include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 can include one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer device 140 of the computer system 300 may use a microphone for voice recognition or an eye tracking camera 366 for tracking eyeball movement. In some implementations, the computer device 140 can include one or more optical cameras (e.g., an RGB camera), scanners, or photo sensor units for capturing images. The computer system 300 may also can include one or more output devices 312 that enable presentation of user interfaces 210 and media content. The one or more output devices 312 can include one or more speakers and/or one or more visual displays.

The computer system 300 can include one or more sensors 360, which further can include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more directional microphones 380. It is noted that the one or more sensors 360 can also be included in the input device 310 and used to collect data to the computer system 300.

Memory 306 can include high-speed random-access memory, such as DRAM, SRAM, DDR RAM, or other random-access solid state memory devices; and, optionally, can include non-volatile memory, such as one or more magnetic disk storage devices, one or more optical disk storage devices, one or more flash memory devices, or one or more other non-volatile solid state storage devices. Memory 306, optionally, can include one or more storage devices remotely located from one or more processing units 302. Memory 306, or alternatively the non-volatile memory within memory 306, can include a non-transitory computer readable storage medium. In some implementations, memory 306, or the non-transitory computer readable storage medium of memory 306, may store the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof can include a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training Module 332 for obtaining training data 346 and training machine learning models 350; and
One or more databases 340 for storing at least data including one or more of:
Device settings 334 including common device settings (e.g., service tier, device model, storage capacity, processing capabilities, communication capabilities, etc.) of the computer system 300;
User account information 336 for the one or more user applications 324, e.g., user names, security questions, account history data, user preferences, and predefined account settings, where in some embodiments, the user account information 336 can include facial measurements and one or more virtual fitting parameters associated with associated with a user account of an eye fitting application 326, and in some embodiments, the user account information 336 can include visual stimuli 338, sensor data 342, and vision test results 344 associated with a user account of a visual assessment application 328; and
Machine learning models 350 including parameters (e.g., weights, biases) used to implement vision test or select eyewear for eyewear users.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in some embodiments. In some embodiments, memory 306, optionally, stores a subset of the modules and data structures identified above. Furthermore, memory 306, optionally, stores additional modules and data structures not described above.

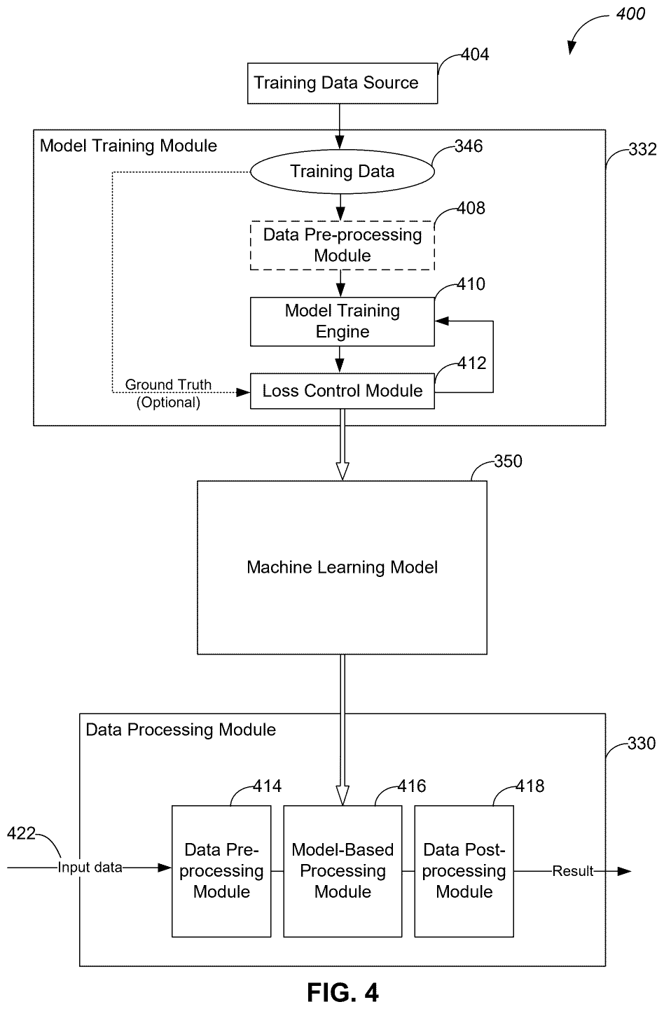

FIG. 4 is a block diagram of a machine learning system 400 for training and applying machine learning models 350 (e.g., for glass making), in accordance with some embodiments. The machine learning system 400 can include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 can include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real-time input data 422 detected by the computer device 140. In some embodiments, the training data 346 provided by the training data source 404 can include a standard dataset widely used to train machine learning models 350. The input data 422 further can include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 can include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criterion (e.g., a comparison result of the loss function is minimized or reduced below a loss threshold). The machine learning models 350 may thereby be trained and provided to the data processing module 330 of a computer device 140 to process real-time input data 422 from the computer device 140.

In some embodiments, the model training module 402 may further can include a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and can include a desired output for each training data item (also called the ground truth, in some embodiments). In some embodiments, the desirable output may be labelled manually by people or automatically by the model training model 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 can include a data pre-processing module 414, a model-based processing module 416, and a data post-processing module 418. The data pre-processing modules 414 may pre-process input data 422 based on the type of the input data 422. In some embodiments, functions of the data pre-processing modules 414 are consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

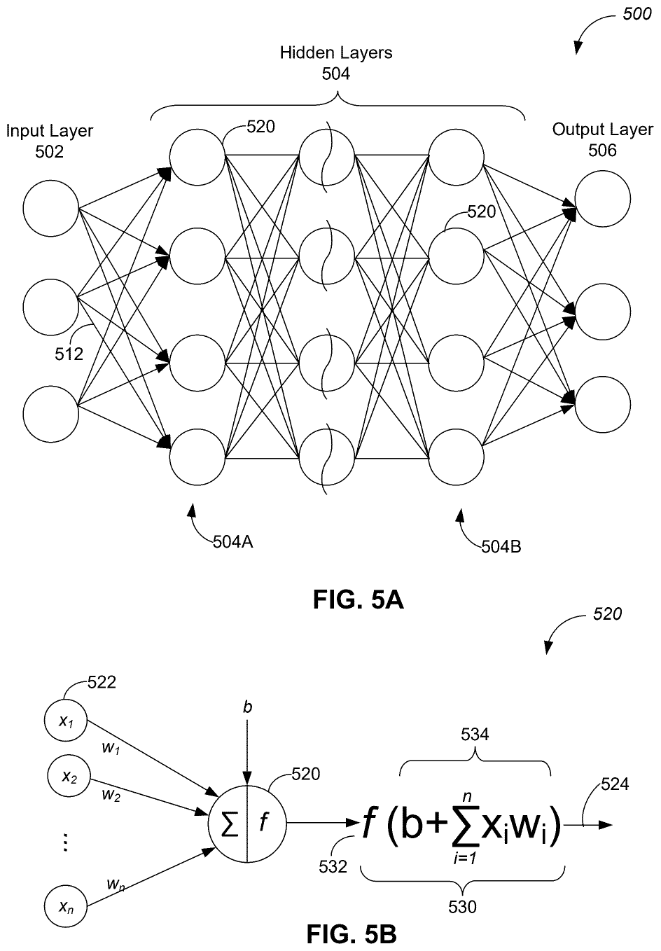

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 can include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers can include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a “fully connected” layer because each node in the layer is connected to every node in its immediately following layer. In some embodiments, a hidden layer 504 can include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN can include convolutional layers. Each node in a convolutional layer may receive inputs from a receptive area associated with a previous layer (e.g., nine nodes). Each convolution layer may use a kernel to combine pixels in a respective area to generate outputs.

For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may be abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights wᵢ for each layer of the neural network 500 using training data 346 that is provided in the input layer 502. The training process typically can include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training can include a network bias parameter b for each layer.

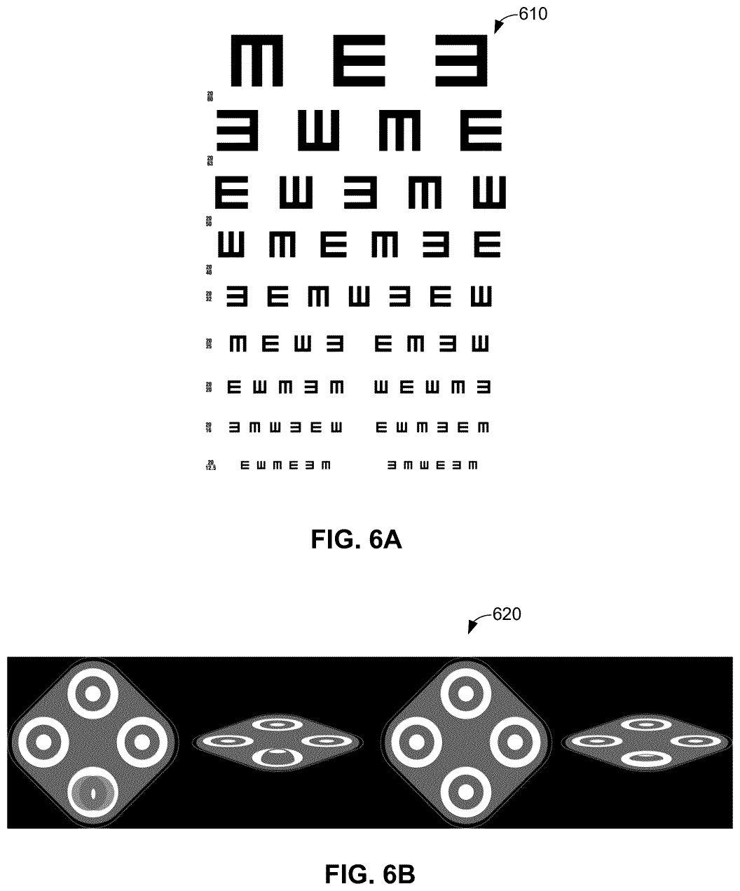

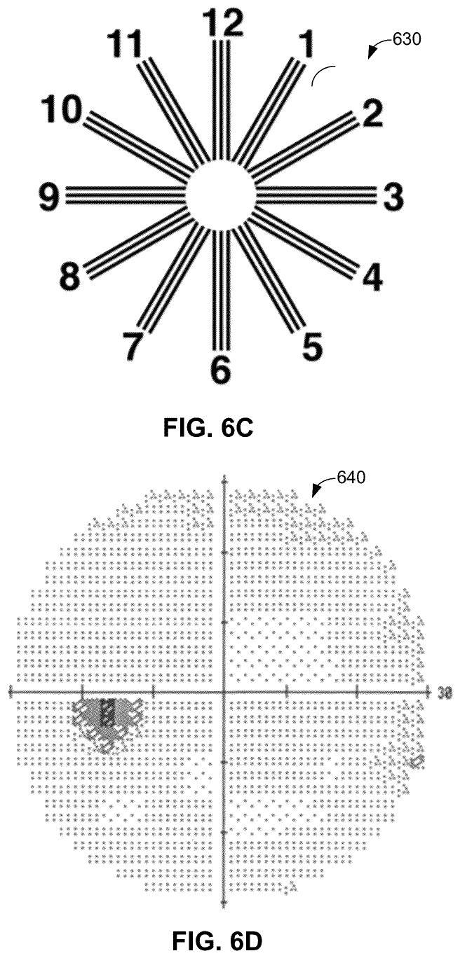

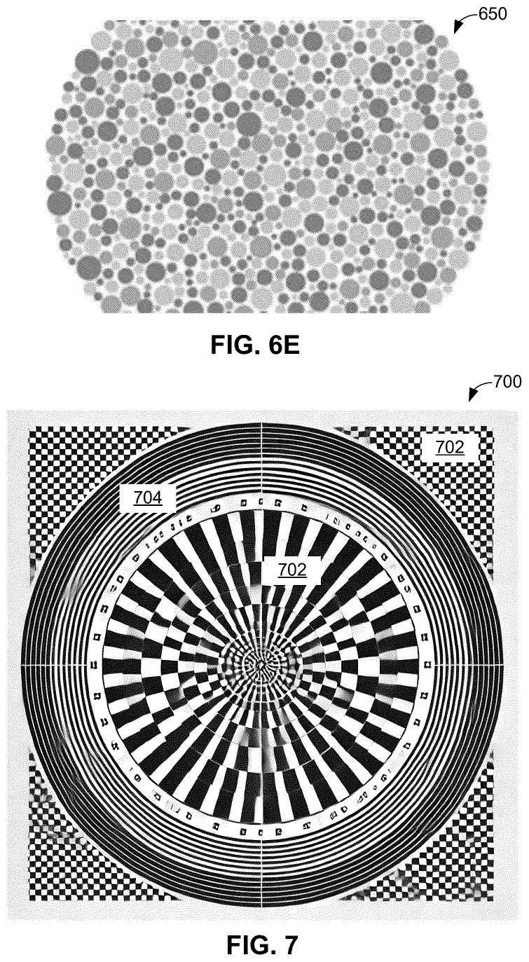

In some embodiments of the present disclosure, a vision test is implemented in a headset device 140D configured to display a user interface creating a three-dimensional (3D) virtual environment. Examples of a vision test implemented in the 3D virtual environment can include, but are not limited to a visual acuity test, a visual field test, a visual depth test, a color blindness test, a retinoscopy, a test for stercopsis, a refraction test, an astigmatism test, and a contact lens exam. FIG. 6A is an example “tumbling E” chart 610 applied in a visual acuity test, in accordance with some embodiments. FIGS. 6B, 6C, 6D, and 6E are example patterns 620, 630, 640, and 650 applied in an astigmatism test, a stereopsis test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 can include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

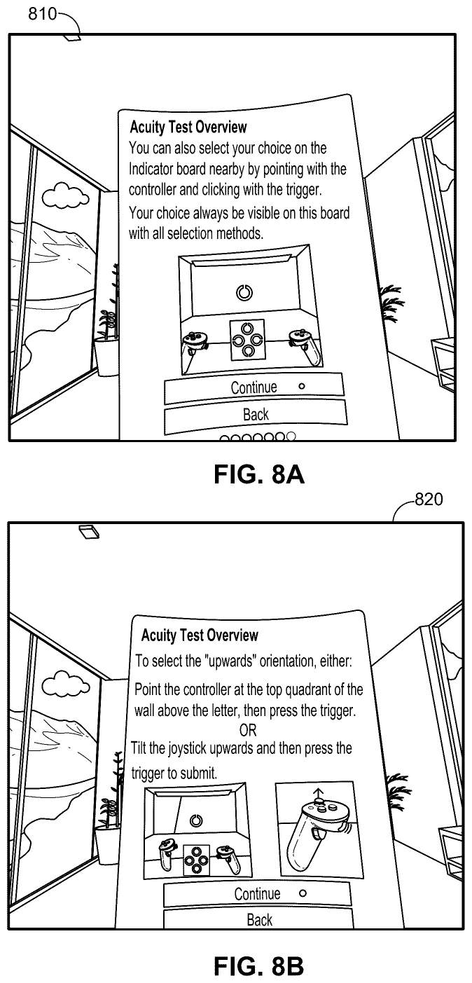

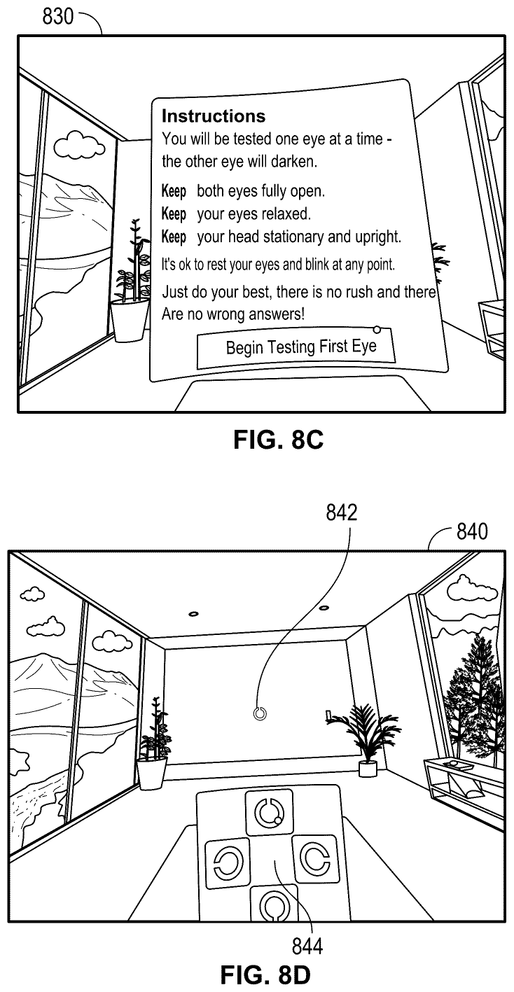

FIGS. 8A-8D can include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using the controller to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

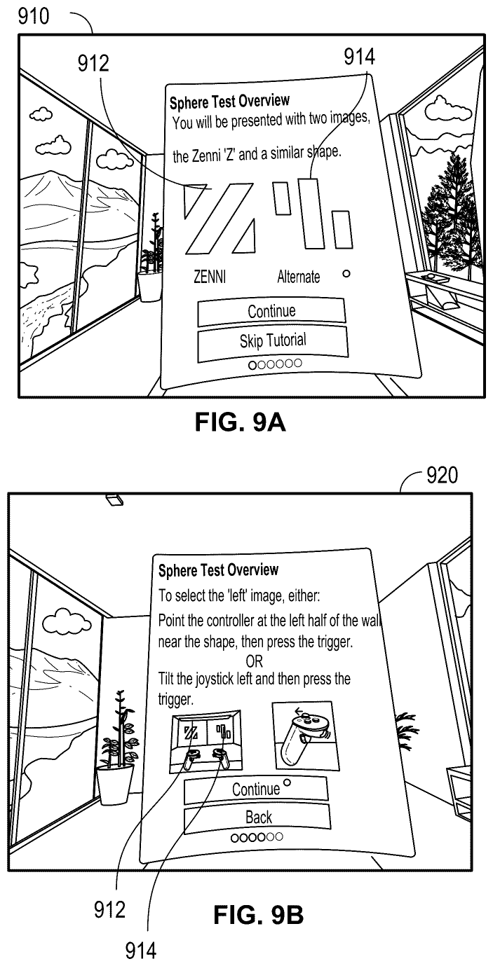

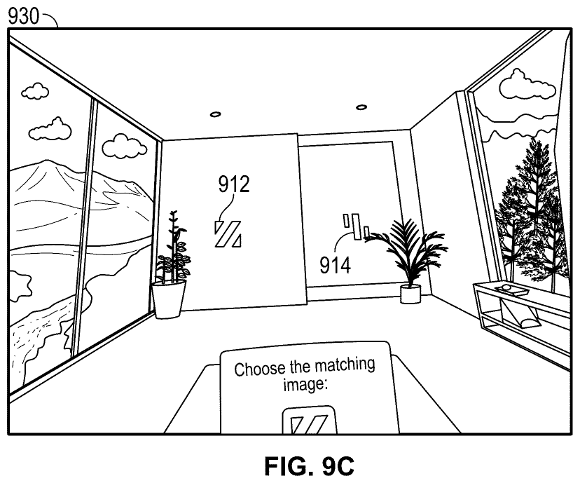

FIGS. 9A-9C can include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using the controller to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel 932 may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

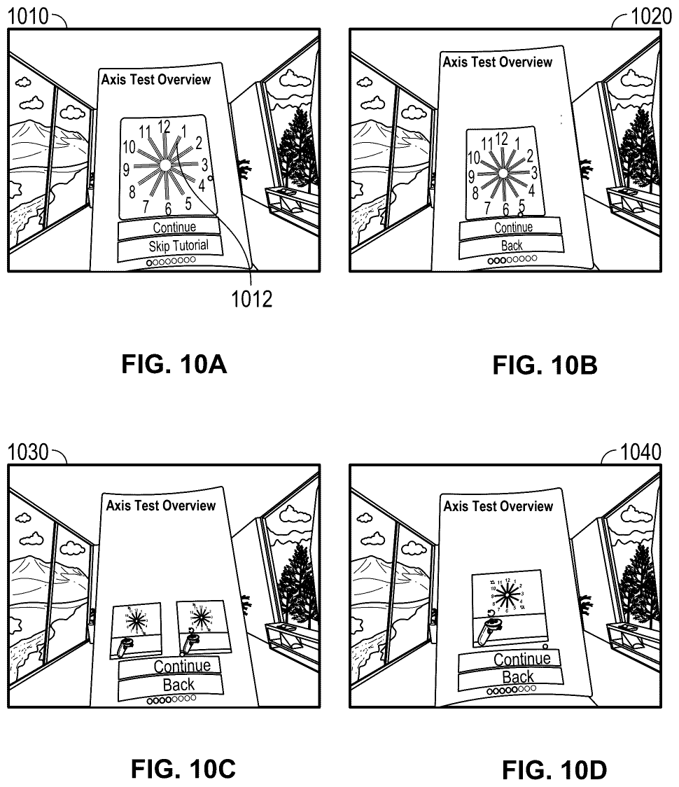

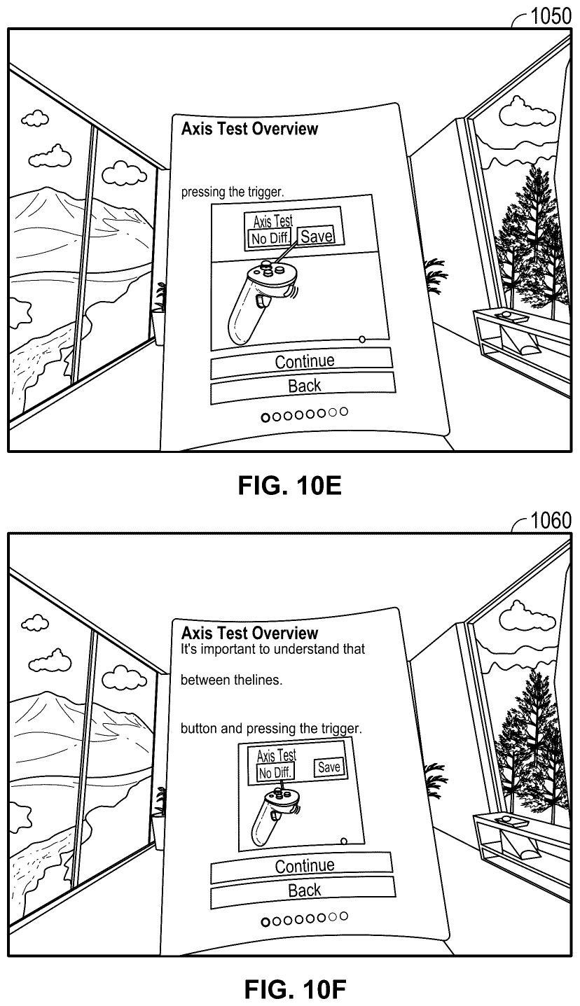

FIGS. 10A-10F can include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 can include a message, e.g., “You will be presented with a clock diagram of converging numbered lines.”

The user interface 1020 may display an information page explaining what is selected on the clock diagram of converging numbered lines 1012 displayed in the virtual environment. For example, the user interface 1010 can include a message, e.g., “Your task is to identify if any of these sets of lines appear clearer, crisper, or darker than other.”

Further, the user interface 1030 may display an information page including two optional ways of using the controller to select lines on the clock diagram of converging numbered lines 1012. For example, the user interface 1010 can include a message, e.g., “Make a selection by either pointing the controller at the lines on the clock, then pressing the trigger” and “Rotating the joystick to move the indicator arrows around the clock.”

The user interface 1040 may display an information page illustrating an embodiment having equally clear lines on the clock diagram of converging numbered lines 1012. For example, the user interface 1010 can include a message, e.g., “If two sets of neighboring lines seem to both stand out as equally clear, you can move the indicator arrows to a halfway point between those lines.”

Referring to FIG. 10E, the user interface 1050 may display an information page including an instruction using the controller to submit a selection. For example, the user interface 1010 can include a message, e.g., “After selecting a set of lines, submit your choice with the ‘Done’ button below by pointing to the controller at the button and pressing the trigger.”

Further, referring to FIG. 10F, the user interface 1060 may display an information page including an instruction using the controller to indicate that no difference is observed on the clock diagram of converging numbered lines 1012. For example, the user interface 1010 can include a message, e.g., “It's important to understand that not everybody will see a difference between the lines” and “In this case, simply select ‘No Difference’ below, by positioning the controller at the button and pressing the trigger.”

### VR System for Evaluating and Improving Vision for Daily Life and Career

This disclosure can include various embodiments of VR systems that can broadly be categorized as VR systems for evaluating and improving vision in aspects of daily life and career. The VR system can include a VR headset in electronic communication with a computing device. The VR headset is worn by a user and can include screens, eye-tracking sensors, and eye-tracking cameras. In some embodiments, the VR headset has a singular screen positioned in front of the user's eyes. In other embodiments, the VR headset has two screens, with each screen being positioned in front of one of the user's eyes. Optionally, the screens in the VR headset are lenses, with one lens being positioned in front of each of the user's eyes.

The eye-tracking sensors and the eye-tracking cameras are configured to monitor the user's eyes, collect data about the user, and communicate that data with the computing device. For example, the eye-tracking sensors and the eye-tracking cameras can be configured to monitor fixation points, reaction times, gaze direction, eye movements, fixation patterns, response times, visual response accuracies, blink rates, fixation stabilities, pupil sizes, eye positions, saccadic movements, and smooth pursuit movements. Optionally, the eye-tracking sensors can comprise ambient light sensors, which are particularly useful for compensating for light seeping into the VR headset and potentially altering the lighting conditions of the vision training or assessment. Optionally, the eye-tracking sensors and the eye-tracking cameras can be infrared.

In some embodiments, the VR headset can include sensors that can be configured to monitor the user's heart rate, skin conductance, and body temperature.

In some embodiments, the VR headset can include microphones for receiving verbal input, speakers for providing auditory feedback to the user, and vibrating motors and heat sensors for providing haptic feedback (e.g., vibrations, changes in temperature, etc.) to the user. Optionally, the speakers, vibrating motors, and heat sensors can be used for making the virtual environment more realistic in addition to motivating the user to continue with the activity in the virtual environment and correcting the user's responses. In other embodiments, the VR headset can include electrodes configured to attach to the user's scalp and monitor the user's brainwave patterns.

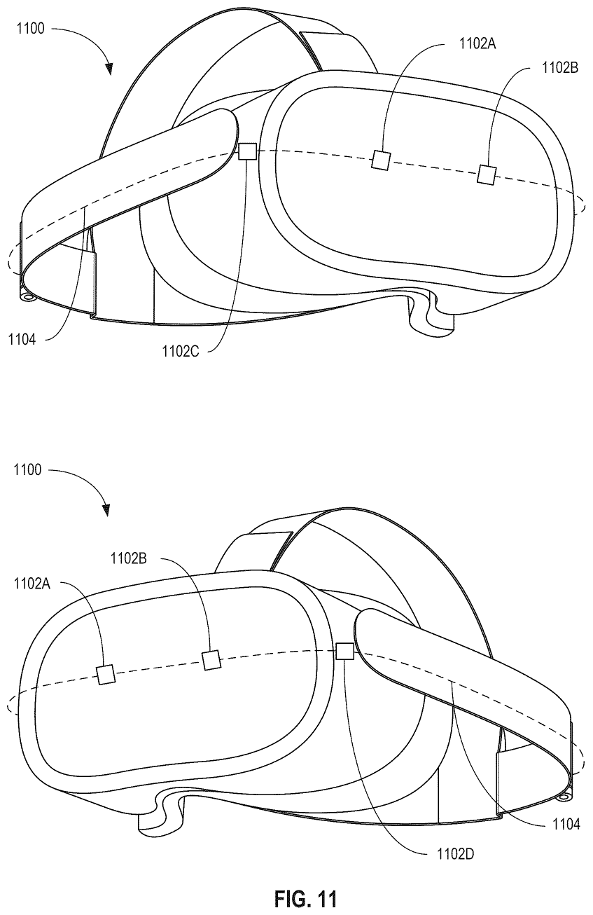

In some embodiments, the VR headset can include motion-tracking sensors. The motion-tracking sensors can be configured to track physical movements and a spatial orientation of the user within the virtual environment. The motion-tracking sensors can be further configured to track a three-dimensional movement of the head of the user to identify the translation, pitch, yaw, and roll of the user's head as she moves around the virtual environment, tests out different furniture arrangements, etc. In some embodiments, the motion-tracking sensors can be configured to track physical movements, spatial orientation, and three-dimensional movement in real-time and communicate the corresponding data to the computing device in real-time. Optionally, the motion-tracking sensors can be configured to track a speed at which the user moves her head in different directions. Optionally, the motion-tracking sensors can comprise accelerometers and/or gyroscopes. The positions of the motion-tracking sensors on the VR headset are depicted in FIG. 11.

FIG. 11 illustrates a placement of motion-tracking sensors on a VR headset, in accordance with some embodiments. FIG. 11 shows two perspective views of a VR headset 1100 with motion-tracking sensors 1102 (1102A, 1102B, 1102C, and 1102D). Each VR headset 1100 can have at least four motion-tracking sensors 1102, with at least two motion-tracking sensors 1102A, 1102B being placed on a front portion of the VR headset 1100, at least one motion-tracking sensor 1102C being placed on a right portion of the VR headset 1100, and at least one motion-tracking sensor 1102D being placed on a left portion of the VR headset 1100. The front portion is the portion of the VR headset 1100 that would be in front of the user's eyes, the right portion would be near the user's right ear, and the left portion would be near the user's left ear.

In some embodiments, the motion-tracking sensors 1102 can be on the outer exterior of the VR headset 1100 (on the surface of the VR headset 1100 that is away from the user's face), on the inner exterior of the VR headset 1100 (on the surface of the VR headset 1100 that is adjacent to or in contact with the user's face), or on the interior of the VR headset 1100. In some embodiments, the placement of the motion-tracking sensors 1102 can be asymmetrical around the user's head.

In some embodiments, the motion-tracking sensors 1102 can be positioned along a transverse plane of the VR headset 1100. The transverse plane can align with the eyeline of the user after or when the user dons the VR headset 1100 and is indicated by dashed line 1104 in FIG. 11.

The computing device is configured to cause virtual environments with different features (e.g., furniture, machinery, landmarks, people, weather, etc.) and various lighting conditions (e.g., clouds, bright sunlight, refracted light underwater, sunset, nighttime, etc.) to be displayed on the screens of the VR headset. Moreover, the computing device can also be configured to cause optotypes, objects, and visual tasks to be displayed in the virtual environment. The computing device can prompt the user to participate in different interactive activities involving the optotypes, objects, and visual tasks. These are described in greater detail below with respect to the different VR methods embodied in this disclosure.

The computing device can also be configured to process the data collected by the VR headset in order to evaluate various aspects of the user's vision and physiological capabilities in light of the different virtual environments. In some embodiments, the computing device can include an advanced algorithm that processes this data to evaluate the user's vision and physiological capabilities. Optionally, the algorithm can process and evaluate in real-time while the user responds to the prompts provided by the computing device.

In some embodiments, the algorithm can change the virtual environments shown on the screens of the VR headset by changing the features and/or lighting conditions in the virtual environments. Moreover, the algorithm can change the optotypes and objects in the virtual environment by changing a size, shape, brightness, contrast, etc. of the optotypes and objects. Similarly, the algorithm can change the nature and/or difficulty of the visual task displayed in the virtual environment. Optionally, the algorithm can implement these changes based on the user's responses (visual or otherwise physiological) to the prompts provided by the computing device. Optionally, the algorithm can implement these changes in real-time while the user responds to the prompts.

Optionally, where the VR system is used to create a customized vision therapy plan for the user, the computing device is further configured to develop the vision therapy plan by processing the user's responses to the prompts provided by the computing device (e.g., the data collected by the VR headset) and by drawing from a library of visual tasks designed to improve a user's vision. Moreover, the computing device can guide the user through the customized vision therapy plan and analyze the user's responses to the various visual tasks to assess the long-term effectiveness of the customized vision therapy plan. Additionally, the computing device can analyze the user's responses and recommend adjustments to the vision therapy plan by changing the visual tasks, the difficulty of the visual tasks, the order of the visual tasks, the frequency with which the user should perform the visual tasks, etc.

Optionally, the algorithm of the computing device can be configured to process the data collected by the VR headset to evaluate a user's visual clarity underwater, depth perception underwater, and ability to adapt to changes in water conditions. In some embodiments, the algorithm can process the data and evaluate the user's underwater vision capabilities in real-time while the user responds to the prompts provided by the computing device.

Optionally, the algorithm of the computing device can be configured to process the user's responses to the prompts provided by the computing device (e.g., the data collected by the VR headset such as eye movements, pupil dilation, heart rate, brain wave patterns, etc.) to evaluate the user's visual clarity, reaction time, visual acuity consistency, and focusing consistency. In some embodiments, the algorithm can process the data and evaluates the boundaries of the user's cognitive workload and the user's current cognitive workload in real-time while the user responds to the prompts provided by the computing device.

Optionally, the algorithm of the computing device can be configured to process the data collected by the VR headset to evaluate a user's visual acuity, depth perception, reaction speed, and peripheral awareness. Using this information the algorithm can adjust the features of the virtual environment as well as the task being conducted in the virtual environment to create an optimal vision and reflex training activity for the user. In some embodiments, the algorithm can process the data and evaluate the user's vision and reflex capabilities in real-time while the user responds to the prompts provided by the computing device.

Optionally, the algorithm of the computing device can be configured to assess an extent to which users follow a predetermined set of rules (e.g., vision safety protocols) while responding to the prompts provided by the computing device. The algorithm can evaluate the rules by assessing the degree to which the users can follow the rules, how efficiently the users work when bound by the rules, and a degree to which the rules accomplish their intended purpose (e.g., do the rules protect the users from hazards in a workplace environment)? Based on the evaluation, the algorithm can adjust the rules, and the computing device can prompt the users to repeat the tasks while following the adjusted rules.

In some embodiments, the VR system can also include a handheld device (e.g., a controller, a handset, a glove, etc.) in electronic communication with the VR headset and the computing device. The handheld device can be configured to receive input from the user as the user follows the prompts provided by the computing device or otherwise engages with the virtual environment. For instance, the user can use the handheld device to type in responses to visual tasks, to point at or grab objects in the virtual environments, to catch or target moving objects, etc.

Optionally, the VR system can include multiple VR headsets worn by multiple users. In some embodiments, these users can work together in the virtual environment to complete tasks. In other embodiments, the users can compete against each other in the virtual environment while completing various tasks. The users can see the exact same virtual environment, or the virtual environment that each user sees can be tailored based on the users' abilities and performance.

### Testing and Training Vision Adaptability Using Simulated Real-World Scenarios

One embodiment of this disclosure is directed to a VR method for assessing a user's vision adaptability. Vision adaptability is a person's ability to accurately perceive the world despite the presence of optical obscurities. Vision adaptability can include a person's ability to guess what objects or components might be missing from her field of view as well as the person's ability to see through or around the obscurities. Testing and training vision adaptability in VR is particularly helpful because VR facilitates the simulation of real-world scenarios, which means the test and the training program can engage multiple groups of eye muscles simultaneously.

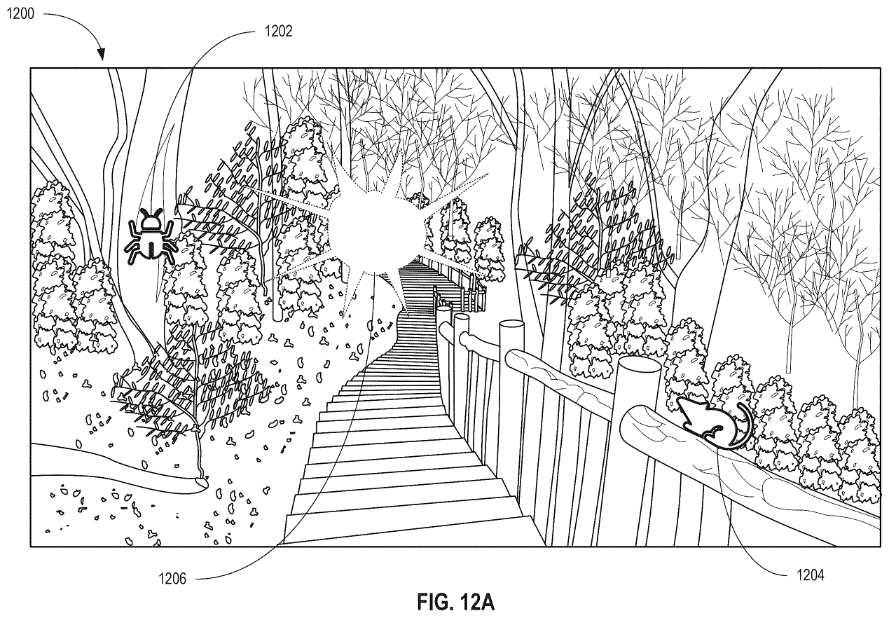

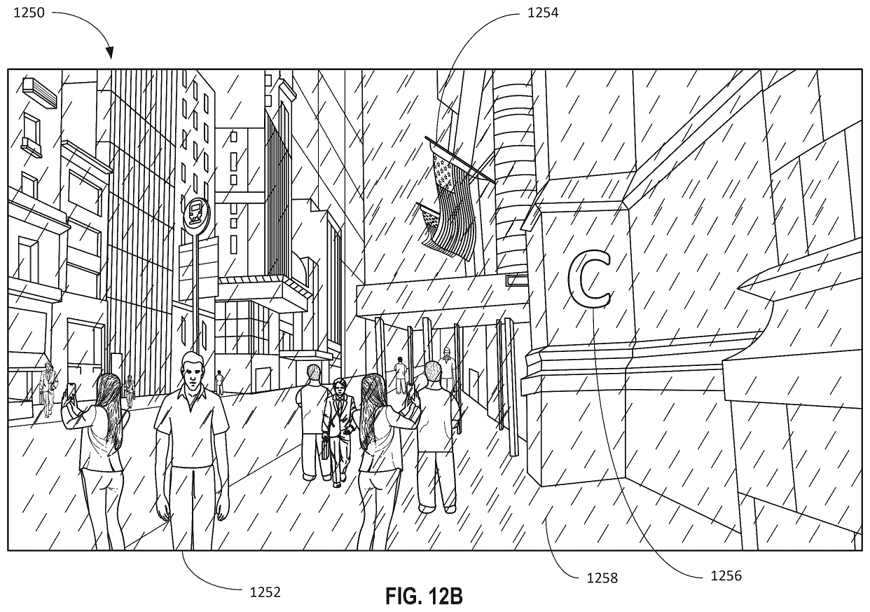

The method begins with the user dons the VR headset. A virtual environment will be displayed on the screens of the VR headset. In some embodiments, the virtual environment can be a driving simulation. The virtual environment can also be a first-person perspective of the user walking along a busy street, working in an office, or walking through a store. Examples of virtual environments are illustrated in FIGS. 12A and 12B.

FIGS. 12A and 12B illustrate virtual environments that simulate real-world scenarios and include real-world obscurities such as glare and rain, in accordance with some embodiments. Specifically, FIG. 12A shows a virtual environment 1200 of a nature hike on a sunny day, and FIG. 12B shows a virtual environment 1250 of a busy city street on a rainy day.

Next, the computing device causes a visual task to be displayed in the virtual environment. The visual task can include targeting objects positioned at different distances from the user in the virtual environment while navigating the environment (e.g., while driving, walking, working, etc. as described above), which induces the user to quickly shift her focus between near and far objects. For example, referring to FIG. 12A, the computing device can prompt the user to shift her focus between the bug 1202, which is far away from the user, and the rat 1204, which is close to the user, in the virtual environment 1200. This type of visual task can be directed towards training and assessing the user's saccadic eye movements as well as the user's focusing ability. The visual task can also include tracking moving objects in the virtual environment or tracking a stationary object as the user walks through the virtual environment. For example, referring to FIG. 12B, the computing device can prompt the user to focus on a person 1252 in the crowd that is walking towards her or to focus on the flag 1254 as she walks down the sidewalk.

Optionally, the objects can move between areas near the user and areas that are far away from the user to induce the user to adapt her vision to different depths of field. In some embodiments, the objects can move quickly to assess and train the user's rapid near/far focusing ability. In other embodiments, the objects can move more slowly to gently guide the user through changing her depth of focus. This type of visual task can be directed toward training and assessing the user's smooth pursuit movements as well as the user's focusing ability.

While the user engages with the visual task and the virtual environment, the VR headset monitors the eye movements of the user, which can include monitoring the user's gaze direction, fixation stability, visual response accuracy, saccadic movements, and smooth pursuit movements. Monitoring saccadic and smooth pursuit movements is particularly helpful because the computing device can analyze this information to identify neurological deficits and muscle imbalances.

The computing device then causes the virtual environment to be changed. This can include adjusting the brightness, contrast, saturation, etc. of the virtual environment. This can also include displaying a day-time environment or a night-time environment (as well as in-between lighting conditions such as dusk or dawn), so that the user's vision adaptability can be tested in different settings. Moreover, this can include displaying various weather conditions in the virtual environment such as a bright sunny day with glare 1206 in the virtual environment 1200 of FIG. 12A or a rainy day with raindrops 1258 in the virtual environment 1250 of FIG. 12B.

Optionally, adjusting the virtual environment can include adjusting objects in the virtual environment. For example, the computing device can change the direction that people in the virtual environment are walking (e.g., the people on the street in the virtual environment 1250 in FIG. 12B), the degree to which leaves in trees are moving (e.g., the leaves in the trees in the virtual environment 1200 in FIG. 12A), a speed at which water in a river is flowing, etc.

Optionally, adjusting the virtual environment can include adjusting the position of objects in the virtual environment. For instance, the computing device can move some or all of the objects closer or farther away from the user, mirror the position of the objects in the virtual environment, rotate objects in the virtual environment, etc. These adjustments can also serve to adjust the distance of the objects on which the user is prompted to focus.

Optionally, the computing device can change various features or conditions of the virtual environment abruptly or between extremes. For example, the computing device will cause the virtual environment to abruptly change from day to night, which tests and trains the user's ability to switch quickly between seeing in broad daylight and seeing in the dark.

Optionally, the computing device can increase a quantity of obscurities displayed in the virtual environment. For example, the computing device can cause the virtual environment to have more rain, hail, or snow (e.g., increasing or decreasing the density of raindrops 1258 displayed in the virtual environment 1250 of FIG. 12B). The virtual environment can also increase an amount of wind displayed in the virtual environment, which would result in more particles flying through the air and obscuring the user's vision of the virtual environment and the virtual task. Moreover, the virtual environment can increase the intensity of glare in the virtual environment (e.g., glare point 1206 in the virtual environment 1200 of FIG. 12A).

In some embodiments, the computing device adjusts the virtual environment and the visual task in real-time based on the user's eye movements and other responses to the visual task and the virtual environment. For example, the adjustments can be made to make the training or testing more difficult for users who are easily completing the visual task. Likewise, the adjustments can be made to make the training or testing easier for users who are struggling in order to keep the user engaged, which makes the training more effective and the testing more accurate. In some embodiments, the computing device can change the difficulty of the assessment or training upon determining that the user has consistently met a baseline threshold of capability.

In some embodiments, the computing device can include an algorithm that processes the user's eye movements and other responses to the visual tasks and the virtual environment to evaluate the user's reaction time, visual acuity, and focus stability. Optionally, the algorithm can process and evaluate in real-time while the user follows the prompts provided by the computing device and responds to the visual tasks and the virtual environment.

The computing device is also configured to compare the user's eye movements and responses to the various iterations of the visual tasks and the virtual environment to a database of eye movements and responses from individuals with known vision adaption capabilities. This helps evaluate the user's vision adaptability.

### Creating Personalized Vision Therapy Sessions

Another embodiment of this disclosure is directed to developing and adapting personalized vision therapy sessions for a user. VR is well-suited to conducting vision therapy sessions because it is convenient to occlude one of a user's eyes in VR, which facilitates individual training of the eyes. This can be helpful when the user's eyes have different problems that need to be corrected. VR also makes quantifying and measuring various factors of the user's eye movements convenient.

The method begins by administering an assessment on the screens of a VR headset worn by the user. The assessment can include vision tests configured to identify various visual deficiencies, such as strabismus, amblyopia, convergence insufficiency, etc.

While the user completes the assessment, the VR headset monitors the user's eye movements. The eye-tracking sensors and the eye-tracking cameras of the VR headset can monitor the user's gaze direction, fixation stability, response accuracy, and response times. In some embodiments, the VR headset is continuously monitoring the user's eye movements and continuously communicating this data to the computing device so the computing device can process it.

Next, the computing device can process the user's eye movements to identify a type and a severity of the user's visual deficiencies (e.g., extremely bad visual acuity, mild to moderate strabismus, etc.). Optionally, the computing device can process the user's eye movements in real-time while the user completes the assessment. This real-time processing can be used to adjust the assessment to obtain more data about areas in which the patient struggles. This real-time processing can also be used to provide the user with results more quickly.

Upon processing the user's eye movements, the computing device references a library of visual tasks (e.g., tracking moving objects, targeting objects, identifying optotypes, navigating through obstacle courses with different weather conditions, etc.) to determine a quantity and order of visual tasks that make up the user's personalized vision therapy plan. Additionally, the computing device can determine the nature of the visual tasks and the difficulty of the visual task. Optionally, the computing device references the library in real-time while the user completes the assessment. This real-time development of the vision therapy plan can be used to provide the user with results more quickly.

This disclosure also includes methods for adapting the personalized vision therapy sessions as the user partakes in the vision therapy sessions. At a first session, the user participates in the visual tasks that make up her personalized vision therapy session. As she completes the visual tasks, the VR headset monitors her eye movements, as described above. The computing device receives the data from the VR headset and processes it to evaluate how successfully the user is completing the tasks, how effective the tasks are with respect to improving the user's vision, etc.

Based on this evaluation, the computing device can adjust the visual tasks. For example, the computing device can change the quantity of visual tasks, the order of the visual tasks, the duration of each visual task (e.g., shortening the tasks if the user is showing signs of exhaustion or lengthening the tasks if the user needs more training), the nature of the visual tasks (e.g., whether the visual tasks exercise the user's eye muscles, induce her to change her blink rate, or guide her through color perception exercises), or the difficulty of the visual tasks (e.g., by changing the speed or the extremes at which objects are displayed in the virtual environment). Optionally, the computing device can implement an algorithm to select and execute these changes. Optionally, the visual tasks are adjusted in real-time during the vision therapy session. Optionally, the visual tasks are adjusted so that the next vision therapy session is made up of the adjusted visual tasks.

### Assessing and Mitigating Accommodative Spasm

Some embodiments of this disclosure are directed to VR methods for assessing and mitigating accommodative spasm (which can also be referred to low accommodative ability or accommodation lock). People with accommodative spasm cannot efficiently change their depth of focus because their eyes are locked into one depth of focus. This problem is increasingly common because people spend a lot of time looking at screens (e.g., phone or tablet screens) very close to their faces. As a result, their eyes become locked into a focal depth that is very close to their face, and seeing objects that are far away becomes very difficult.

A method for assessing accommodative spasm begins when the computing device causes a virtual environment to be displayed on the screens of a VR headset worn by the user. The virtual environment can include visually demanding scenarios such as driving, navigating through a crowd of people, working in a hospital, navigating through a natural disaster, etc. The virtual environment can also include scenarios with prolonged screen exposure such as working in an office or scenarios where the user is exposed to glare or other lighting conditions that obscure vision.

The computing device also causes one or more objects to be displayed in the virtual environment. The objects can be an array of optotypes, and the computing device can prompt the user to identify optotypes in the array. The objects can also be singular optotypes or three-dimensional objects of varying sizes (water bottles, chair, cars, buildings, etc.).

Optionally, the computing device can change the background color of the virtual environments. For example, where the object is an array of black optotypes, the computing device can change the background color of the array from red to green (or vice versa). Because the red background will result in a sharper virtual environment, if the user does not change her focus, then this is a clear indicator of accommodative spasm.

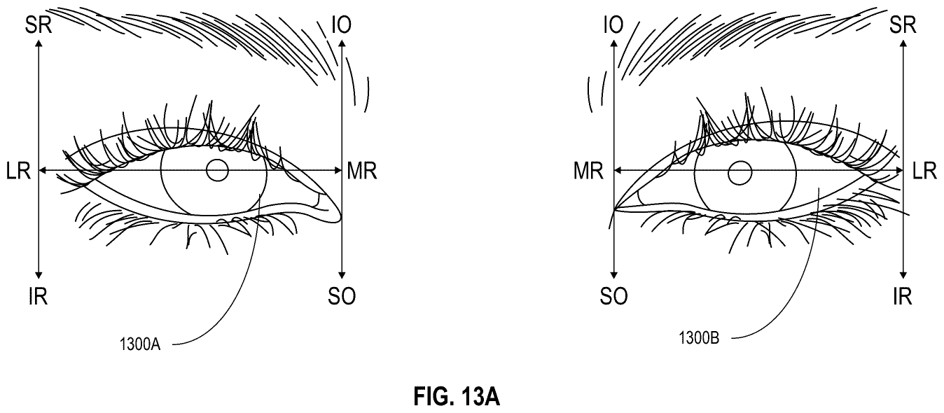

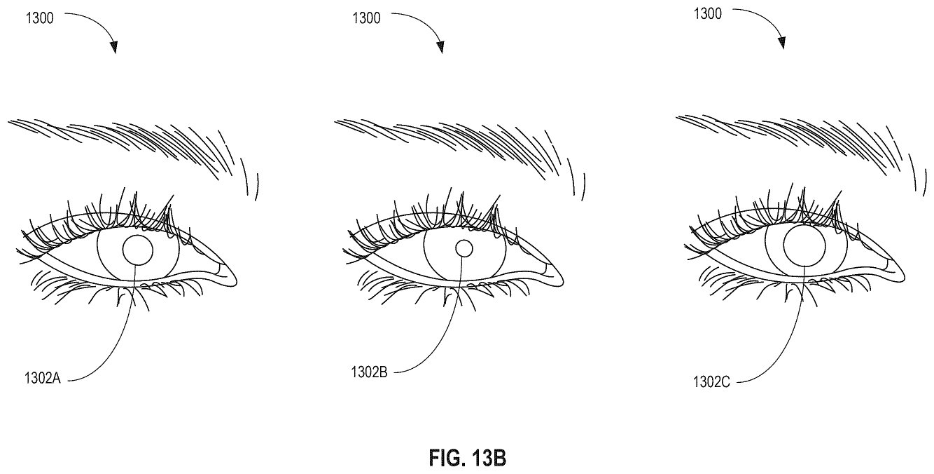

While the user focuses on the one or more objects in accordance with the prompt from the computing device, the VR headset monitors the user's eye position and pupil size because the user's eye position and pupil size can be used to determine whether the user is focusing on the correct point; such a scenario is described in greater detail below with respect to FIGS. 13A and 13B. Using data about the user's eye position and pupil size, the computing device can calculate an exact depth and position at which the user's eyes are focus and, thus, determine whether the user is focused on the object that she was told to focus on. If the user has accommodative spasm, the computing device might notice that her eye position and pupil size do not properly adjust to the correct depth and position of focus.

FIG. 13A illustrates charts of the cardinal gaze positions labeled with the extraocular muscles that correspond with the gaze positions superimposed over the user's eyes, in accordance with some embodiments. FIG. 13A shows charts 1302A and 1302B over the right eye 1300A and the left eye 1300B, respectively. The charts 1302A, 1302B are an “H” diagram of the different directions in which the eyes 1300A, 1300B can look, and the charts 1302A, 1302B are labeled with the extraocular muscles that correspond with those directions.

Every person must use different eye muscles to move her eyes in different directions. Specifically, the superior rectus (SR) is a muscle on top of the eye that moves the eye upward; the inferior rectus (IR) is a muscle on the bottom of the eye that moves the eye downward; the medial rectus (MR) is a muscle on the portion of the eye that is near the nose and moves the eye inward (toward the nose); the lateral rectus (LR) is a muscle on the portion of the eye that is near the car and moves the eye outward (toward the car); the superior oblique (SO) is a muscle that starts at the back of the eye socket, passes by the nose, and attaches to the top of the eye to rotate the eye inward, move the eye downward, and move the eye outward; and the inferior oblique (IO) is a muscle that starts at the front of the eye socket near the nose and attaches to the bottom of the eye to rotate the eye outward, move the eye upward, and move the eye outward. Each of these muscles is labeled in the charts in FIG. 13A.

When the user is focusing on an object that is close to her, her eyeballs 1300A, 1300B will turn in towards her nose, which is an eye movement that largely relies on the medial rectus. When the object is close to the user in the virtual environment, the eye-tracking sensors and the eye-tracking cameras will expect to perceive the eyeballs 1300A, 1300B turning in towards each other. In contrast, when the user is focusing on an object that is far away from her, her eyeballs 1300A, 1300B will turn out towards her cars, which largely involves use of the lateral rectus muscles. When the object is far away from the user in the virtual environment, the eye-tracking sensors and the eye-tracking cameras of the VR headset will expect to perceive the eyeballs 1300A, 1300B turning out towards the user's ears (i.e., towards the “LR” portion of the chart).

FIG. 13B illustrates a normal pupil, a constricted pupil, and a dilated pupil, as perceived by a VR headset, in accordance with some embodiments. As shown, the eye 1300A has a pupil 1302A in a natural, relaxed state. When the user is focusing on an object that is close to her, her pupil will constrict or shrink. As a result, the eye-tracking sensors and eye-tracking cameras of the VR headset may perceive that the user's eye 1300 has a constricted pupil 1302B when the object in the virtual environment is close to the user. On the other hand, when the user is focusing on an object that is far away from her in the virtual environment, her pupil will dilate or become larger. Consequently, the eye-tracking sensors and eye-tracking cameras of the VR headset may perceive that the user's eye 1300 has a dilated pupil 1302C when the object in the virtual environment is far away from the user.

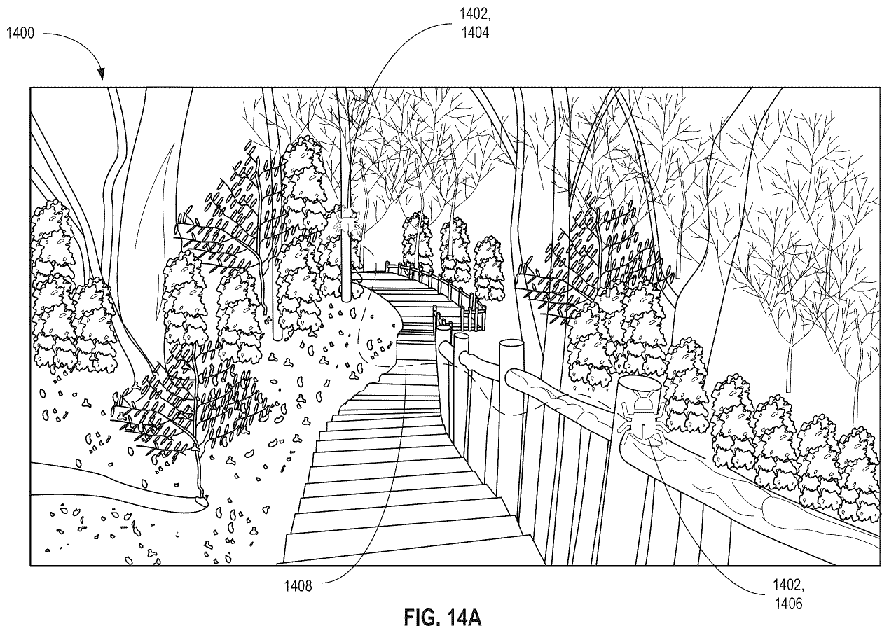

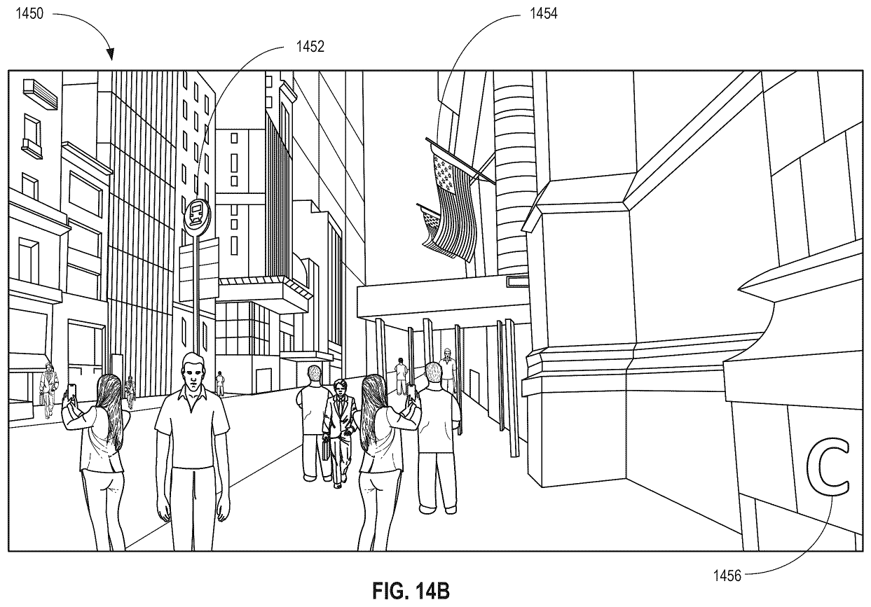

When the computing device determines that the user is focused on the object, the computing device causes the object to be displayed at a different position within the virtual environment and prompts the user to focus on the object at the different position. Preferably, the object is now positioned a different distance away from the user so that the user has to adjust her depth of focus to focus on the object; such a scenario is illustrated in FIG. 14A.

FIG. 14A illustrates a virtual environment of a forest with a bug displayed in two different positions, in accordance with some embodiments. FIG. 14A shows a virtual environment 1400 of a forest with a bug 1402 at a first position 1404 and a second position 1406. The computing device can prompt the user to focus on the bug 1402 at the first position 1404 and then the second position 1406 (or vice versa) to evaluate whether the user can change her depth of focus. In some embodiments, the bug 1402 can move from the first position 1404 to the second position 1406 by disappearing from the virtual environment 1400 at the first position 1404 and reappearing at the second position 1402. In other embodiments, the computing device causes the bug 1402 to move along the path 1408 from the first position 1404 to the second position 1406. The path 1408 can be a straight line, one or more smooth curves, or a jagged line.

Optionally, the computing device can move the object to a different position and prompts the user to focus on a different object in the virtual environment instead of or in addition to moving the original object; such a scenario is shown in FIG. 14B.

FIG. 14B illustrates a virtual environment of a busy street with various objects at different distances from the user, in accordance with some embodiments. In particular, FIG. 14B shows a virtual environment 1450 of a busy city street with various objects such as the bus sign 1452, the flag 1454, and the optotype 1456 at different distances away from the user. The computing device can prompt the user to focus on something far away such as the bus sign 1452 and, once the computing device determines that the user is focused on the object, the computing device can prompt the user to focus on something close to her such as the optotype 1456.

The VR headset monitors the user's eyes to give data to the computing device. This data includes the user's eye position, pupil size, gaze direction, blink rate, and fixation stability. The computing device can use this information to determine whether the user is focused on the object she was told to focus on (or an extent to which she is focused on the object) and how long it takes her to switch her focus. In some embodiments, the computing device determines that the user cannot switch her depth of focus.

Optionally, the computing device can change the virtual environment and the objects within the virtual environment. This is described in greater detail above with respect to the VR method for assessing a user's vision adaptability.

This disclosure also includes methods for mitigating accommodative spasm. This method begins when the VR system shows a virtual environment, such as the ones described above, on the screens of the VR headset.

Next, the computing device provides the users with prompts and guidance to complete various eye relaxation exercises. These eye relaxation exercises can include the 20-20-20 rule or variations of it. The 20-20-20 rule says that for every 20 minutes that a person stares at a screen, she should spend 20 seconds looking at something 20 feet away. The computing device can also prompt the user to focus her gaze on an object in the virtual environment that is approximately 5-15 feet away, 15-25 feet away, 25-35 feet away, or 35-45 feet away. This can be valuable if the user does not have space in her home to focus on objects that are farther away or if the user does not have objects in her workplace that are the correct distance away from her. Optionally, the computing device can adapt the eye relaxation exercises based on the objects and the amount of space that the user has in her home or her workplace.

In a different embodiment, the eye relaxation exercises can include prompting the user to track an object that is moving around the virtual environment. For example, with reference to FIG. 14A, the bug 1402 might fly along the path 1408 from the first position 1404 to the second position 1406. The bug 1402 can fly through different parts of the user's depth of field at different speeds. By prompting the user to visually track the bug 1402 (or other objects in a virtual environment), the computing device induces the user to become accustomed to changing the depth of her gaze and relaxing her eyes out of accommodative spasm.

In some embodiments, the computing device recommends and teaches the user how to do eye relaxation exercises outside of the VR system. For example, the computing device might recommend that the user palm her eyes with her hands.

Optionally, the VR headset provides the user with auditory, tactile, and visual cues to help guide the user through the eye relaxation exercises. For example, the VR headset might play verbal instructions, buzz to get the user's attention when she is not engaging with the exercises, or display written instructions on the screens.

### Conducting Occupational Vision Testing Using Simulated Underwater Environments

Another embodiment of this disclosure is a VR method for evaluating the user's underwater vision. This method is centered around displaying a simulated underwater environment on the VR headset. The computing device can change the brightness and contrast of the environment to simulate different depths and clarities of water. The computing device can also vary a density of particles in the environment to simulate different clarities of water and warp rays of light that shine through the underwater environment to simulate light being refracted by water and by various objects or particles in the water.

When the user is immersed in the simulated underwater environment, the computing device administers visual tasks for the user. The visual task can include identifying or grabbing objects that are positioned at different distances away from the user and at different depths in the water, using tools to collect objects or catch animals that are in the water, navigating around obstacles in the water (e.g., navigating through a coral reef or through underwater caves), navigating through different lighting conditions in the water (e.g., through different depths of water, in and out of underwater caves, etc.), prompting a user to follow moving objects or animals through the water, etc. These visual tasks can also be combined and changed to be more or less difficult or to be tailored to specific occupations.

While the user participates in the visual task, the VR headset monitors her eye movements. The eye-tracking sensors and eye-tracking cameras can monitor the user's gaze direction, fixation stability, response accuracy, saccadic movements, and smooth pursuit movements-among other aspects, which are described above with respect to the eye-tracking sensors and cameras.

Using this information, the computing device can evaluate the user's visual clarity, depth perception, and ability to adapt to light, temperature, and pressure underwater. The computing device can also use the information obtained from the VR headset to change the difficulty of the visual task. Optionally, the computing device can comprise an algorithm that can process the information collected by the VR headset, evaluate the user's ability to complete tasks underwater, and adjust the difficulty or nature of the visual task.

Optionally, the computing device can compare the user's eye movement data, visual clarity, depth perception, and ability to adapt to changing underwater conditions to a database with information about people who are deemed qualified to participate in occupations that require underwater vision capabilities.

Optionally, the simulated underwater environment can be used to train the user and prepare her for carrying out tasks underwater.

### Testing Cognitive Workload in Response to Changing Weather

Some embodiments of this disclosure are directed to VR methods for testing a user's cognitive workload as weather conditions in her environment change. This method begins by displaying a virtual environment for the user on the screens of the VR headset. The virtual environment can resemble the virtual environments depicted in FIGS. 12A and 12B, described above. The features of the virtual environment can also be changed as described above with respect to the VR method for testing and training vision adaptability in real-world scenarios.

Next, the computing device causes a visual task to be displayed in the virtual environment. In addition to the visual tasks described above with respect to the VR method for testing and training vision adaptability in real-world scenarios, the visual task in this embodiment can also include navigating through virtual environments with reduced visibility (e.g., due to low lighting, fog, smoke, etc.), navigating through a maze, and navigating through an obstacle course. Optionally, the visual task can include collecting objects positioned at different distances from the user, which requires the user to both navigate through the environment and identify the object.

While the visual task is being displayed, the computing device also causes one or more weather features to be displayed in the virtual environment, and the user has to complete the visual task under different weather circumstances. Weather features might include bright sunlight (which can result in bright lighting or glare points such as the glare 1206 in the virtual environment 1200 of FIG. 12A), fog, rain (such as the raindrops 1258 in the virtual environment 1250 of FIG. 12B), snow, wind, smoke, hurricanes, tornadoes, floods, etc. Optionally, the computing device can change the weather features in between visual tasks or while the user is completing any given visual task.

The VR headset can monitor the user while she completes the visual task to collect information about her eye movements, pupil dilation, heart rate, and brain wave patterns. The pupil dilation, heart rate, and brain wave patterns provide important information about the user's physiological state.

In some embodiments, the computing device can receive and process the information collected by the VR headset to tailor the difficulty and nature of the visual task to the user. Additionally, the computing device can process the information to evaluate the user's visual clarity, her reaction time, the consistency of her visual acuity, and the consistency of her focusing ability, among other things. The computing device can also process this information to evaluate the user's cognitive workload. In other embodiments, the computing device can include an algorithm that completes these analyses and evaluation. Optionally, the algorithm can process and evaluate in real-time while the user follows the prompts provided by the computing device and otherwise responds to the visual tasks and the virtual environment.

### Improving Vision and Reflexes in Response to Multifactorial Demands

A different embodiment of the present disclosure is directed to a VR method for improving vision and reflexes. This is particularly useful in scenarios where the user might be exposed to multifactorial demands such as while working at a hospital, driving, or playing sports. The method begins by displaying a virtual environment on the screens of the VR headset. As described above with regard to other embodiments in this disclosure, the brightness, contrast, saturation, weather, etc. of the virtual environment can be changed.

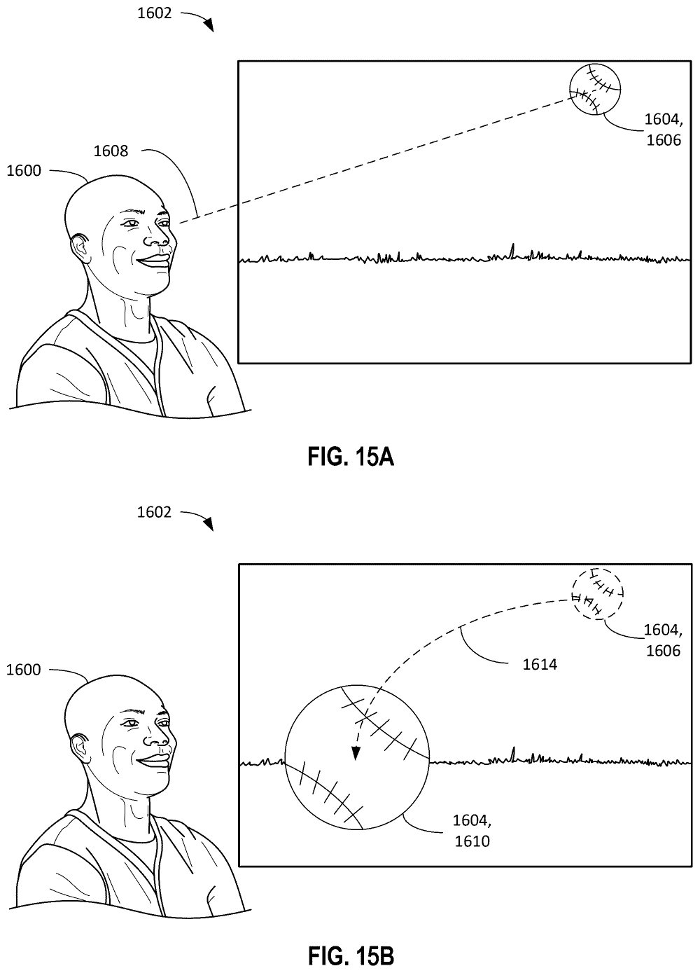

Once the virtual environment is being displayed, an object is displayed in the virtual environment. The object can move quickly around the virtual environment at a certain speed and along a certain trajectory. The speed and trajectory of the object can be changed as the user engages with the virtual environment and the object. In some embodiments, the object is a ball that the user must catch or hit, as shown in FIGS. 15A and 15B, described in greater detail below. In other embodiments, the object is a person or animal that the user must catch or tag. In some embodiments, the object is something that the user must dodge or avoid. The object can move at any speed within the environment, but in some embodiments, the object can move between 30 mph and 100 mph.

FIGS. 15A and 15B illustrate a baseball flying towards a user in a VR environment, in accordance with some embodiments. As shown, the user 1500 is viewing a virtual environment 1502. In FIG. 15A, the baseball 1504 is at a first position 1506 that is far away from the user. In FIG. 15B, the baseball 1504 has traveled to the second position 1510 along a trajectory 1514. The second position 1510 is much closer to the user than the first position 1506.

Next, the user is prompted to interact with the object. This can include running towards the object and catching it, catching or grabbing the object, predicting the trajectory of the object, locating the object, tracking the object with her eyes, etc. For example, with reference to FIGS. 15A and 15B, the user might be prompted to catch the baseball 1504 when it is at the second position 1510 or hit the baseball 1504 with a bat. Optionally, the baseball 1504 can move from the second position 1510 to the first position 1506, and the user might be prompted to chase the baseball 1504.

In some embodiments, one or more objects are shown in the virtual environment, and the user is prompted to switch between focusing on the different objects. Optionally, where two objects are shown in the virtual environment, the user can be prompted to visually fuse the two objects together into a single object. Optionally, the user can use a handheld device to interact with the object (e.g., catch or hit a ball).

While the user interacts with the object (or objects) in the virtual environment, the VR headset monitors the user's eyes, as described above with respect to the other methods disclosed in this application. For instance, with reference to FIGS. 15A and 15B, the VR headset can monitor the user's eyes to determine the exact position of the user's gaze. For example, the eye-tracking sensors and cameras can observe the user's gaze direction 1508 so that the computing device can determine whether the user is focused on the baseball 1504 at the first position 1506. The eye-tracking sensors and cameras can also be used to calculate an extent to which the user's gaze position is incorrect (e.g., the user is always looking two degrees to the left of the ball or always looking an inch below the ball).

Using the information obtained from the VR headset, the computing device can process the information and determine whether and how much to change the speed and/or trajectory of the object. In other embodiments, the computing device can include an algorithm that completes these analyses and evaluation. Optionally, the algorithm can process and evaluate in real-time while the user follows the prompts provided by the computing device and otherwise responds to the visual tasks and the virtual environment.

The speed and trajectory of the object can be tailored to different sports. The tasks that the user must complete in the environment can also be tailored to different sports.

### Evaluating Vision Safety Protocols Using Simulated Occupational Hazards

In this embodiment of the disclosure, a VR method is used to evaluate vision safety protocols in construction sights, factories, hospitals, and other workplaces. The method begins by displaying a virtual environment on the screens of the VR headset. Next, various visual hazards are displayed in the virtual environment. Some examples of visual hazards include extremely bright lights (which can obscure the user's vision), flying debris, and high-speed machinery.

Next the user is provided with a set of rules or vision safety protocols and prompted to complete a task while following the vision safety protocols. The vision safety protocols can include wearing protective eyewear, having a minimum visual acuity requirement, etc. The task can include navigating a vehicle through a construction site, operating heavy machinery, responding to healthcare emergencies, and using a tool to grab an object in the virtual environment. Other tasks in other fields of work are also possible.

While the user completes the task, the VR headset monitors the user's actions and eye movements. Specifically, the VR headset can monitor the user's gaze direction, fixation stability, and reaction time. Using this information, the computing device can assess a degree to which the user followed the vision safety protocols and a degree to which the user successfully and efficiently completed the task. Optionally, the VR headset continuously monitors the user's actions and eye movements, and the computing device completes this assessment in real-time as the user executes the task.

The computing device can also process the information collected by the VR headset to evaluate the efficacy of the vision safety protocols. For example, if the user could not complete the task successfully while following the vision safety protocols, then the computing device can indicate (e.g., via a report) that the vision safety protocols must be changed.

Optionally, the computing device can analyze the user's performance and the vision safety protocols and cross reference this with evaluations of functional and efficient safety protocols in other businesses or areas of industry to recommend changes to the vision safety protocols. In some embodiments, the computing device repeats the method to evaluate the efficacy of the new vision safety protocols. In other embodiments, this process is repeated until the computing device develops a set of optimized vision safety protocols.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for assessing vision adaptability, the method comprising displaying a virtual environment on screens of a virtual reality (VR) headset worn by a user; administering a visual task in the virtual environment at a first time; monitoring first eye movements of the user while the user completes the visual task at the first time; adjusting a condition of the virtual environment; administering the visual task in the virtual environment at a second time; monitoring second eye movements of the user while the user completes the visual task at the second time; and comparing one or more of the first and second eye movements to a database to assess a user's vision adaptability.

Clause 2. The method of any of the preceding Clauses, wherein displaying a virtual environment comprises displaying a driving simulation.

Clause 3. The method of any of the preceding Clauses, wherein displaying a virtual environment comprises displaying a first-person perspective of the user walking along a street.

Clause 4. The method of any of the preceding Clauses, wherein administering a visual task comprises prompting the user to target one or more objects in the virtual environment, wherein each of the one or more objects is positioned at a different distance from the user.

Clause 5. The method of any of the preceding Clauses, wherein administering a visual task comprises prompting the user to track one or more objects that are moving in the virtual environment.

Clause 6. The method of any of the preceding Clauses, wherein monitoring the first and second eye movements of the user comprises monitoring a gaze direction of the user.

Clause 7. The method of any of the preceding Clauses, wherein monitoring the first and second eye movements of the user comprises monitoring a fixation stability of the user.

Clause 8. The method of any of the preceding Clauses, wherein monitoring the first and second eye movements of the user comprises monitoring a visual response accuracy of the user.

Clause 9. The method of any of the preceding Clauses, wherein monitoring the first and second eye movements of the user comprises monitoring saccadic movements of the user.

Clause 10. The method of any of the preceding Clauses, wherein monitoring the first and second eye movements of the user comprises monitoring smooth pursuit movements of the user.

Clause 11. The method of any of the preceding Clauses, wherein adjusting the condition of the virtual environment comprises implementing a software to dynamically adjust the condition of the virtual environment.

Clause 12. The method of any of the preceding Clauses, wherein adjusting the condition of the virtual environment comprises adjusting a brightness of the virtual environment.

Clause 13. The method of any of the preceding Clauses, wherein adjusting the condition of the virtual environment comprises adjusting a contrast of the virtual environment.

Clause 14. The method of any of the preceding Clauses, wherein adjusting the condition of the virtual environment comprises displaying a day-time environment.

Clause 15. The method of any of the preceding Clauses, wherein adjusting the condition of the virtual environment comprises displaying a night-time environment.

Clause 16. The method of any of the preceding Clauses, wherein adjusting the condition of the virtual environment comprises adjusting a movement of one or more objects in the virtual environment.

Clause 17. The method of any of the preceding Clauses, wherein adjusting the condition of the virtual environment comprises adjusting a position of one or more objects in the virtual environment.

Clause 18. The method of any of the preceding Clauses, further comprising processing the first eye movement in real-time as the user completes the visual task at the first time to adjust a difficulty level of the visual task administered at the second time.

Clause 19. The method of any of the preceding Clauses, further comprising processing the first and second eye movements in real-time as the user completes the visual task at the first and second times to evaluate a reaction time of the user.

Clause 20. The method of any of the preceding Clauses, further comprising processing the first and second eye movements in real-time as the user completes the visual task at the first and second times to evaluate a visual acuity of the user.

Clause 21. The method of any of the preceding Clauses, further comprising processing the first and second eye movements in real-time as the user completes the visual task at the first and second times to evaluate a focus stability of the user.

Clause 22. The method of any of the preceding Clauses, further comprising providing auditory and haptic feedback to the user while the user completes the visual task at the first and second times.

Clause 23. The method of any of the preceding Clauses, further comprising recommending adaptive training exercises to improve the user's vision adaptability.

Clause 24. A method for training vision adaptability, the method comprising: displaying a virtual environment on screens of a virtual reality (VR) headset worn by a user; administering a visual task in the virtual environment at a first time; monitoring first eye movements of the user while the user completes the visual task at the first time; adjusting a condition of the virtual environment to increase a difficulty of the visual task; administering the visual task in the virtual environment at a second time with the difficulty increased; and monitoring second eye movements of the user while the user completes the visual task at the second time.

Clause 25. The method of Clause 24, wherein administering the visual task in the virtual environment comprises administering a series of increasingly challenging visual tasks in the virtual environment.

Clause 26. The method of any of Clauses 24 through 25, wherein displaying a virtual environment comprises displaying a driving simulation.

Clause 27. The method of any of Clauses 24 through 26, wherein displaying a virtual environment comprises displaying a first-person perspective of the user walking along a street.

Clause 28. The method of any of Clauses 24 through 27, wherein administering a visual task comprises prompting the user to target one or more objects in the virtual environment, wherein each of the one or more objects is positioned at a different distance from the user.

Clause 29. The method of any of Clauses 24 through 28, wherein administering a visual task comprises prompting the user to track one or more objects that are moving in the virtual environment.

Clause 30. The method of any of Clauses 24 through 29, wherein monitoring the first and second eye movements of the user comprises monitoring a gaze direction of the user.

Clause 31. The method of any of Clauses 24 through 30, wherein monitoring the first and second eye movements of the user comprises monitoring a fixation stability of the user.

Clause 32. The method of any of Clauses 24 through 30, wherein monitoring the first and second eye movements of the user comprises monitoring a visual response accuracy of the user.

Clause 33. The method of any of Clauses 24 through 32, wherein monitoring the first and second eye movements of the user comprises monitoring saccadic movements of the user.

Clause 34. The method of any of Clauses 24 through 33, wherein monitoring the first and second eye movements of the user comprises monitoring smooth pursuit movements of the user.

Clause 35. The method of any of Clauses 24 through 34, wherein adjusting the condition of the virtual environment comprises implementing a software to dynamically adjust the condition of the virtual environment.

Clause 36. The method of any of Clauses 24 through 35, wherein adjusting the condition of the virtual environment comprises implementing a software to process the first eye movement of the user in real-time as the user completes the visual task at the first time and increase the difficulty of the visual task at the second time based on a performance of the user.

Clause 37. The method of any of Clauses 24 through 36, wherein adjusting the condition of the virtual environment comprises adjusting a brightness of the virtual environment.

Clause 38. The method of any of Clauses 24 through 37, wherein adjusting the condition of the virtual environment comprises adjusting a contrast of the virtual environment.

Clause 39. The method of any of Clauses 24 through 38, wherein adjusting the condition of the virtual environment comprises displaying a day-time environment.

Clause 40. The method of any of Clauses 24 through 39, wherein adjusting the condition of the virtual environment comprises displaying a night-time environment.

Clause 41. The method of any of Clauses 24 through 40, wherein adjusting the condition of the virtual environment comprises abruptly changing the condition between two extremes.

Clause 42. The method of any of Clauses 24 through 41, wherein adjusting the condition of the virtual environment comprises increasing a quantity of obscurities displayed in the virtual environment.

Clause 43. The method of any of Clauses 24 through 42, wherein adjusting the condition of the virtual environment comprises increasing a speed of obscurities moving in the virtual environment.

Clause 44. The method of any of Clauses 24 through 43, further comprising processing the first and second eye movements in real-time as the user completes the visual task at the first and second times to evaluate a reaction time of the user.

Clause 45. The method of any of Clauses 24 through 44, further comprising processing the first and second eye movements in real-time as the user completes the visual task at the first and second times to evaluate a visual acuity of the user.

Clause 46. The method of any of Clauses 24 through 45, further comprising processing the first and second eye movements in real-time as the user completes the visual task at the first and second times to evaluate a focus stability of the user.

Clause 47. The method of any of Clauses 24 through 46, further comprising providing auditory and haptic feedback to the user while the user completes the visual task at the first and second times.

Clause 48. The method of any of Clauses 24 through 47, wherein the method is adapted for a multiple-user mode such that the method comprises: simultaneously displaying the virtual environment on the screens of multiple VR headsets worn by multiple users; simultaneously administering the visual task in the virtual environment for each user of the multiple users; monitoring the first eye movements of each user of the multiple users while each user completes the visual task at the first time; adjusting the condition of the virtual environment to increase the difficulty of the visual task; administering the visual task in the virtual environment for each user of the multiple users at the second time with the difficulty increased; and monitoring the second eye movements of each user of the multiple users while each user completes the visual task at the second time.

Clause 49. A system for testing and training vision adaptability, the system comprising: a virtual reality (VR) headset worn by a user, the VR headset comprising screens, one or more eye-tracking sensors, and one or more eye-tracking cameras, wherein the one or more eye-tracking sensors and cameras are configured to collect eye movement data from the user; and a computing device in electronic communication with the VR headset, the computing device being configured to display a virtual environment on the screens of the VR headset, administer visual tasks in the virtual environment, and adjust conditions of the virtual environment, wherein the eye movement data comprises a user's responses to the virtual environment and the visual tasks.

Clause 50. The system of Clause 49, further comprising a handheld device in electronic communication with the VR headset and the computing device, wherein the handheld device is configured to receive input from the user as the user completes the visual task.

Clause 51. The system of any of Clauses 49 through 50, wherein the one or more eye-tracking sensors comprise ambient light sensors.

Clause 52. The system of any of Clauses 49 through 51, wherein the one or more eye-tracking cameras comprise infrared cameras.

Clause 53. The system of any of Clauses 49 through 52, wherein the eye movement data comprises fixation points of the user while the user completes the visual tasks.

Clause 54. The system of any of Clauses 49 through 53, wherein the eye movement data comprises reaction times of the user while the user completes the visual tasks.

Clause 55. The system of any of Clauses 49 through 54, wherein the VR headset further comprises speakers and vibrating motors, wherein the speakers and the vibrating motors are configured to provide auditory and haptic feedback to the user while the user completes the visual tasks.

Clause 56. The system of any of Clauses 49 through 55, further comprising a different VR headset in electronic communication with the VR headset and the computing device, wherein the different VR headset comprises screens, sensors, and cameras, the sensors and cameras being configured to collect eye movement data from a different user.

Clause 57. The system of Clause 56, wherein the computing device is configured to display the virtual environment on the screens of the different VR headset, administer visual tasks in the virtual environment for the different user, and adjust the conditions of the virtual environment.

Clause 58. A method for adapting personalized vision therapy sessions, the method comprising: at a first session: administering a series of visual tasks on screens of a virtual reality (VR) headset worn by a user, wherein the series of visual tasks comprises a quantity of visual tasks, an order of visual tasks, a nature of at least one visual task, and a difficulty of the at least one visual task; monitoring a user's eye movements while the user completes the series of visual tasks; processing the user's eye movements to analyze a user's vision progress; and adjusting the series of visual tasks based on the user's vision progress; and at a second session, administering the adjusted series of visual tasks.

Clause 59. The method of Clause 58, wherein administering the series of visual tasks comprises displaying moving objects in a virtual environment and prompting the user to track the moving objects.

Clause 60. The method of any of Clauses 58 through 59, wherein administering the series of visual tasks comprises displaying objects at varying distances from the user and prompting the user to target the objects.

Clause 61. The method of any of Clauses 58 through 60, wherein administering the series of visual tasks comprises administering each visual task in the series of visual tasks in order of increasing difficulty.

Clause 62. The method of any of Clauses 58 through 61, wherein monitoring the user's eye movements comprises monitoring a user's gaze direction.

Clause 63. The method of any of Clauses 58 through 62, wherein monitoring the user's eye movements comprises monitoring a user's fixation stability.

Clause 64. The method of any of Clauses 58 through 63, wherein monitoring the user's eye movements comprises monitoring a user's response accuracy.

Clause 65. The method of any of Clauses 58 through 64, wherein monitoring the user's eye movements comprises monitoring a user's response times.

Clause 66. The method of any of Clauses 58 through 65, wherein processing the user's eye movements comprises processing the user's eye movements in real-time as the user completes the series of visual tasks.

Clause 67. The method of any of Clauses 58 through 66, wherein adjusting the series of visual tasks comprises changing the quantity of visual tasks.

Clause 68. The method of any of Clauses 58 through 67, wherein adjusting the series of visual tasks comprises changing the order of visual tasks.

Clause 69. The method of any of Clauses 58 through 68, wherein adjusting the series of visual tasks comprises changing the nature of the at least one visual task.

Clause 70. The method of any of Clauses 58 through 69, wherein adjusting the series of visual tasks comprises changing the difficulty of the at least one visual task.

Clause 71. The method of Clause 70, wherein changing the difficulty of the at least one visual task comprises changing a speed of a moving object when the at least one visual task comprises tracking the moving object.

Clause 72. The method of Clause 70, wherein changing the difficulty of the at least one visual task comprises changing a size of an object when the at least one visual task comprises targeting the object.

Clause 73. The method of Clause 70, wherein changing the difficulty of the at least one visual task comprises changing a duration of the at least one visual task.

Clause 74. The method of any of Clauses 58 through 73, wherein adjusting the series of visual tasks comprises changing at least two of the quantity of visual tasks, the order of visual tasks, the nature of the at least one visual task, and the difficulty of the at least one visual task.

Clause 75. The method of any of Clauses 58 through 74, further comprising changing the difficulty of the at least one visual task in real-time as the user completes the series of visual tasks.

Clause 76. The method of any of Clauses 58 through 75, further comprising: monitoring eye movements of multiple users wearing multiple VR headsets while the multiple users complete the series of visual tasks; and storing data about the eye movements in a database.

Clause 77. The method of Clause 76, wherein processing the user's eye movements comprises comparing the user's eye movements to the data about the eye movements in the database.

Clause 78. The method of any of Clauses 58 through 77, further comprising providing auditory and haptic feedback to the user while the user completes the series of visual tasks.

Clause 79. The method of any of Clauses 58 through 78, further comprising generating a summary of the user's vision progress.

Clause 80. A method for developing a personalized vision therapy session, the method comprising: administering an assessment on screens of a virtual reality (VR) headset worn by a user, wherein the assessment comprises a series of vision tests configured to identify visual deficiencies; monitoring a user's eye movements while the user completes the assessment; processing the user's eye movements to identify a type and a severity of a user's visual deficiencies; accessing a library of visual tasks; and based on the type and the severity of the user's visual deficiencies, determining a quantity, an order, a nature, and a difficulty of the visual tasks from the library that constitute the personalized vision therapy session.

Clause 81. The method of Clause 80, wherein administering the assessment comprises administering the series of vision tests configured to identify strabismus.

Clause 82. The method of any of Clauses 80 through 81, wherein administering the assessment comprises administering the series of vision tests configured to identify amblyopia.

Clause 83. The method of any of Clauses 80 through 82, wherein administering the assessment comprises administering the series of vision tests configured to identify convergence insufficiency.

Clause 84. The method of any of Clauses 80 through 83, wherein monitoring the user's eye movements comprises monitoring a user's gaze direction.

Clause 85. The method of any of Clauses 80 through 84, wherein monitoring the user's eye movements comprises monitoring a user's fixation stability.

Clause 86. The method of any of Clauses 80 through 85, wherein monitoring the user's eye movements comprises monitoring a user's response accuracy.

Clause 87. The method of any of Clauses 80 through 86, wherein monitoring the user's eye movements comprises monitoring a user's response times.

Clause 88. The method of any of Clauses 80 through 87, wherein processing the user's eye movements comprises processing the user's eye movements in real-time as the user completes the assessment.

Clause 89. The method of any of Clauses 80 through 88, wherein determining the quantity, the order, the nature, and the difficulty of the visual tasks from the library that constitute the personalized vision therapy session comprises implementing a software to generate the personalized vision therapy session.

Clause 90. A system for creating a vision therapy plan, the system comprising: a virtual reality (VR) headset worn by a user, the VR headset comprising screens, one or more eye-tracking sensors, and one or more eye-tracking cameras; and a computing device in electronic communication with the VR headset, the computing device being configured to administer an assessment on the screens of the VR headset and develop a vision therapy plan for the user by processing a user's responses to the assessment and accessing a library of visual tasks, wherein the one or more eye-tracking sensors and cameras are configured to collect the user's responses to the assessment.

Clause 91. The system of Clause 90, further comprising a handheld device in electronic communication with the VR headset and the computing device, wherein the handheld device is configured to receive input from the user as the user completes the visual task.

Clause 92. The system of any of Clauses 90 through 91, wherein the one or more eye-tracking sensors comprise ambient light sensors.

Clause 93. The system of any of Clauses 90 through 92, wherein the one or more eye-tracking cameras comprise infrared cameras.

Clause 94. The system of any of Clauses 90 through 93, wherein the VR headset further comprises speakers and vibrating motors, wherein the speakers and the vibrating motors are configured to provide auditory and haptic feedback to the user while the user completes the visual tasks.

Clause 95. The system of any of Clauses 90 through 94, wherein the user's responses comprise a gaze direction of the user.

Clause 96. The system of any of Clauses 90 through 95, wherein the user's responses comprise eye movements of the user.

Clause 97. The system of any of Clauses 90 through 96, wherein the user's responses comprise fixation patterns of the user.

Clause 98. The system of any of Clauses 90 through 97, wherein the user's responses comprise response times of the user.

Clause 99. The system of any of Clauses 90 through 98, wherein the user's responses comprise a response accuracy of the user.

Clause 100. The system of any of Clauses 90 through 99, wherein the computing device is further configured to generate a summary of a user's vision progress, and the computing device comprises a user interface at which the summary can be accessed.

Clause 101. The system of any of Clauses 90 through 100, wherein the computing device is further configured to assess a long-term effectiveness of the personalized vision therapy plan and generate recommendations for adjustments to the vision therapy plan.

Clause 102. A method for assessing accommodative spasm, the method comprising: displaying a virtual environment on screens of a virtual reality (VR) headset worn by a user; displaying an object at a first position in the virtual environment; prompting the user to focus on the object at the first position; monitoring a first eye position and a first pupil size of the user as the user focuses on the object in the first position; displaying the object at a second position in the virtual environment, wherein the second position has a different depth in the virtual environment than the first position; prompting the user to focus on the object at the second position, wherein the second position is a different distance away from the user than the first position; monitoring a second eye position and a second pupil size of the user as the user focuses on the object in the second position, wherein the user transitions from the first eye position to the second eye position and from the first pupil size to the second pupil size in response to the object being displayed at the second position; and comparing the first eye position to the second eye position and the first pupil size to the second pupil size to calculate an accommodation amplitude of the user and identify the presence of the accommodative spasm.

Clause 103. The method of Clause 102, wherein displaying the virtual environment comprises simulating visually demanding scenarios.

Clause 104. The method of any of Clauses 102 through 103, wherein displaying the virtual environment comprises simulating prolonged screen exposure.

Clause 105. The method of any of Clauses 102 through 104, wherein displaying the virtual environment comprises simulating glare.

Clause 106. The method of any of Clauses 102 through 105, wherein displaying the virtual environment comprises simulating high-contrast settings.

Clause 107. The method of any of Clauses 102 through 106, wherein displaying the object comprises displaying an array of optotypes, and prompting the user to focus on the object comprises prompting the user to identify optotypes in the array of optotypes.

Clause 108. The method of any of Clauses 102 through 107, further comprising monitoring a gaze direction of the user.

Clause 109. The method of any of Clauses 102 through 108, further comprising monitoring a blink rate of the user.

Clause 110. The method of any of Clauses 102 through 109, further comprising monitoring a fixation stability of the user.

Clause 111. The method of any of Clauses 102 through 110, further comprising analyzing the first eye position and the first pupil size in real-time as the user focuses on the object in the first position.

Clause 112. The method of Clause 111, further comprising, in response to analyzing the first eye position and the first pupil size, determining the second position.

Clause 113. The method of Clause 111, further comprising, in response to analyzing the first eye position and the first pupil size, changing the virtual environment.

Clause 114. The method of any of Clauses 102 through 113, further comprising providing auditory feedback to the user via the VR headset while the user focuses on the object.

Clause 115. The method of any of Clauses 102 through 114, further comprising providing haptic feedback to the user via the VR headset while the user focuses on the object.

Clause 116. The method of any of Clauses 102 through 115, further comprising:
monitoring eye positions and pupil sizes of multiple users wearing multiple VR headsets while the multiple users focus on objects in virtual environments displayed on the screens of the multiple VR headsets; and
storing data about the eye positions and the pupil sizes in a database.

Clause 117. The method of Clause 116, further comprising comparing the first eye position, the first pupil size, the second eye position, and the second pupil size to the data in the database.

Clause 118. A method for mitigating accommodative spasm, the method comprising: displaying a virtual environment on screens of a virtual reality (VR) headset worn by a user; guiding the user through eye relaxation exercises; monitoring an eye position and a pupil size of the user as the user participates in the eye relaxation techniques; processing the eye position and the pupil size of the user; and in response to processing the eye position and the pupil size of the user, adjusting the eye relaxation techniques.

Clause 119. The method of Clause 118, wherein displaying the virtual environment comprises simulating visually demanding scenarios.

Clause 120. The method of Clause 119, wherein simulating visually demanding scenarios comprises gradually increasing an extent to which the visually demanding scenarios are visually demanding.

Clause 121. The method of any of Clauses 118 through 120, wherein displaying the virtual environment comprises simulating prolonged screen exposure.

Clause 122. The method of any of Clauses 118 through 121, wherein displaying the virtual environment comprises simulating glare.

Clause 123. The method of any of Clauses 118 through 122, wherein displaying the virtual environment comprises simulating high-contrast settings.

Clause 124. The method of any of Clauses 118 through 123, wherein displaying the virtual environment comprises simulating an 8-hour workday by displaying various office tasks and prompting the user to complete the various office tasks.

Clause 125. The method of any of Clauses 118 through 124, wherein guiding the user through the eye relaxation techniques comprises prompting the user to focus her gaze on an object in the virtual environment positioned between approximately 5 to 15 feet away from the user for set periods of time.

Clause 126. The method of any of Clauses 118 through 125, wherein guiding the user through the eye relaxation techniques comprises prompting the user to focus her gaze on an object in the virtual environment positioned between approximately 15 to 25 feet away from the user for set periods of time.

Clause 127. The method of any of Clauses 118 through 126, wherein guiding the user through the eye relaxation techniques comprises prompting the user to focus her gaze on an object in the virtual environment positioned between approximately 25 to 35 feet away from the user for set periods of time.

Clause 128. The method of any of Clauses 118 through 127, wherein guiding the user through the eye relaxation techniques comprises prompting the user to focus her gaze on an object in the virtual environment positioned between approximately 35 to 45 feet away from the user for set periods of time.

Clause 129. The method of any of Clauses 118 through 128, wherein guiding the user through the eye relaxation techniques comprises prompting the user to track an object in the virtual environment as a distance between the user and the object changes.

Clause 130. The method of any of Clauses 118 through 129, wherein guiding the user through the eye relaxation techniques comprises providing auditory cues via the VR headset.

Clause 131. The method of any of Clauses 118 through 130, wherein guiding the user through the eye relaxation techniques comprises providing tactile cues via the VR headset.

Clause 132. The method of any of Clauses 118 through 131, wherein guiding the user through the eye relaxation techniques comprises providing visual cues on the screens of the VR headset.

Clause 133. The method of any of Clauses 118 through 132, further comprising monitoring a gaze direction of the user.

Clause 134. The method of any of Clauses 118 through 133, further comprising monitoring a blink rate of the user.

Clause 135. The method of any of Clauses 118 through 134, further comprising monitoring a fixation stability of the user.

Clause 136. The method of any of Clauses 118 through 135, further comprising providing auditory feedback to the user while the user participates in the eye relaxation techniques.

Clause 137. The method of any of Clauses 118 through 136, further comprising providing haptic feedback to the user while the user participates in the eye relaxation techniques.

Clause 138. A system for assessing accommodative spasm, the system comprising: a virtual reality (VR) headset worn by a user, the VR headset comprising screens, one or more eye-tracking sensors, and one or more eye-tracking cameras, wherein the one or more eye-tracking sensors and the one or more eye-tracking cameras are configured to monitor the eye position and pupil size of the user; and a computing device in electronic communication with the VR headset, the computing device being configured to administer an accommodative spasm assessment by causing an object to be displayed at different locations within a virtual environment displayed on the screens of the VR headset and process the eye position and the pupil size of the user to calculate an accommodative amplitude of the user and to identify the presence of the accommodative spasm.

Clause 139. The system of Clause 138, wherein the one or more eye-tracking sensors comprise ambient light sensors.

Clause 140. The system of any of Clauses 138 through 139, wherein the one or more eye-tracking cameras comprise infrared cameras.

Clause 141. The system of any of Clauses 138 through 140, wherein the one or more eye-tracking sensors and the one or more eye-tracking cameras are further configured to monitor a gaze direction of the user.

Clause 142. The system of any of Clauses 138 through 141, wherein the one or more eye-tracking sensors and the one or more eye-tracking cameras are further configured to monitor a blink rate of the user.

Clause 143. The system of any of Clauses 138 through 142, wherein the one or more eye-tracking sensors and the one or more eye-tracking cameras are further configured to monitor a fixation stability of the user.

Clause 144. The system of any of Clauses 138 through 143, wherein the VR headset further comprises speakers and vibrating motors, wherein the speakers and the vibrating motors are configured to provide auditory and haptic feedback to the user while the user responds to the virtual environment and the object.

Clause 145. The system of any of Clauses 138 through 144, wherein the computing device is further configured to analyze the eye position and the pupil size of the user in real-time as the user responds to the virtual environment and the object and to adjust a difficulty of the accommodative spasm assessment based on how successfully the user is responding to the virtual environment and the object.

Clause 146. The system of any of Clauses 138 through 145, wherein the computing device is further configured to generate a report analyzing the effectiveness of the relaxation techniques and highlighting improvements in a user's visual stress management, and the computing device comprises a user interface at which the report can be accessed.

Clause 147. The system of any of Clauses 138 through 146, wherein the computing device is further configured to assess a long-term effectiveness of the eye relaxation techniques and generate recommendations for adjustments to the eye relaxation techniques.

Clause 148. A method for evaluating a user's underwater vision, the method comprising: displaying a simulated underwater environment on screens of a VR headset worn by a user; administering a visual task in the simulated underwater environment; monitoring a user's eye movements while the user completes the visual task; and comparing the user's eye movements to a database to evaluate the user's underwater vision.

Clause 149. The method of Clause 148, wherein displaying the simulated underwater environment comprises varying brightness in the simulated underwater environment to simulate different depths and clarities of water.

Clause 150. The method of any of Clauses 148 through 149, wherein displaying the simulated underwater environment comprises varying contrast in the simulated underwater environment to simulate different depths and clarities of water.

Clause 151. The method of any of Clauses 148 through 150, wherein displaying the simulated underwater environment comprises varying a density of particles in the simulated underwater environment to simulate different clarities of water.

Clause 152. The method of any of Clauses 148 through 151, wherein displaying the simulated underwater environment comprises warping rays of light in the simulated underwater environment to simulate light being refracted by water.

Clause 153. The method of any of Clauses 148 through 152, wherein administering the visual task comprises prompting the user to identify objects in the simulated underwater environment, wherein the objects are positioned at different distances away from the user and at different depths of the simulated underwater environment.

Clause 154. The method of any of Clauses 148 through 153, wherein administering the visual task comprises prompting the user to grab objects in the simulated underwater environment, wherein the objects are positioned at different distances away from the user and at different depths of the simulated underwater environment.

Clause 155. The method of any of Clauses 148 through 154, wherein administering the visual task comprises prompting the user to use a tool to collect objects in the simulated underwater environment, wherein the objects are positioned at different distances away from the user and at different depths of the simulated underwater environment.

Clause 156. The method of any of Clauses 148 through 155, wherein administering the visual task comprises prompting the user to navigate around obstacles in the simulated underwater environment.

Clause 157. The method of any of Clauses 148 through 156, wherein administering the visual task comprises prompting the user to navigate through different lighting conditions in the simulated underwater environment.

Clause 158. The method of any of Clauses 148 through 157, wherein administering the visual task comprises prompting the user to follow a moving object through the simulated underwater environment.

Clause 159. The method of any of Clauses 148 through 158, wherein monitoring the user's eye movements comprises monitoring a user's gaze direction.

Clause 160. The method of any of Clauses 148 through 159, wherein monitoring the user's eye movements comprises monitoring a user's fixation stability.

Clause 161. The method of any of Clauses 148 through 160, wherein monitoring the user's eye movements comprises monitoring a user's response accuracy.

Clause 162. The method of any of Clauses 148 through 161, wherein monitoring the user's eye movements comprises monitoring a user's saccadic movements.

Clause 163. The method of any of Clauses 148 through 162, wherein monitoring the user's eye movements comprises monitoring a user's smooth pursuit movements.

Clause 164. The method of any of Clauses 148 through 163, further comprising processing the user's eye movements to adjust a difficulty of the visual task.

Clause 165. The method of any of Clauses 148 through 164, further comprising processing the user's eye movements in real-time as the user completes the visual task to adjust a difficulty of the visual task while the user is completing the visual task.

Clause 166. The method of Clause 165, wherein processing the user's eye movements comprises evaluating a user's visual clarity underwater.

Clause 167. The method of Clause 165, wherein processing the user's eye movements comprises evaluating a user's depth perception underwater.

Clause 168. The method of Clause 165, wherein processing the user's eye movements comprises evaluating a user's ability to adapt to changes in water conditions.

Clause 169. The method of any of Clauses 148 through 168, further comprising: simultaneously displaying the simulated underwater environment on the screens of multiple VR headsets worn by multiple users; simultaneously administering the visual task in the simulated underwater environment for each user of the multiple users; monitoring the eye movements of each user of the multiple users while each user completes the visual task; and comparing the user's eye movements to the eye movements of each user of the multiple users.

Clause 170. The method of Clause 169, further comprising displaying each user of the multiple users in the simulated underwater environment.

Clause 171. The method of Clause 169, further comprising adjusting features of the simulated underwater environment displayed on the screens of each VR headset of the multiple VR headsets.

Clause 172. The method of any of Clauses 148 through 171, further comprising providing auditory feedback to the user while the user completes the visual task and navigates the simulated underwater environment.

Clause 173. The method of any of Clauses 148 through 172, further comprising providing haptic feedback to the user while the user completes the visual task and navigates the simulated underwater environment.

Clause 174. The method of any of Clauses 148 through 173, further comprising generating a report summarizing a user's performance of the underwater tasks and identifying areas for improvement.

Clause 175. A system for testing vision in simulated underwater environments, the method comprising: a virtual reality (VR) headset worn by a user, the VR headset comprising screens, one or more eye-tracking sensors, and one or more eye-tracking cameras, the one or more eye-tracking sensors and the one or more eye-tracking cameras configured to monitor eye movements, fixation patterns, and blink rates of the user; and a computing device in electronic communication with the VR headset, the computing device being configured to cause a simulated underwater environment to be displayed on the screens, to cause a visual task to be displayed in the simulated underwater environment, and to process the eye movements, the fixation patterns, and the blink rates of the user.

Clause 176. The system of Clause 175, further comprising one or more motion-tracking sensors configured to track physical movements and a spatial orientation of the user, wherein the computing device is further configured to process the physical movements and the spatial orientation of the user.

Clause 177. The system of any of Clauses 175 through 176, further comprising a handheld device in electronic communication with the VR headset and the computing device, wherein the handheld device is configured to receive input from the user as the user completes the visual task.

Clause 178. The system of any of Clauses 175 through 177, wherein the one or more eye-tracking sensors comprise ambient light sensors.

Clause 179. The system of any of Clauses 175 through 178, wherein the one or more eye-tracking cameras comprise infrared cameras.

Clause 180. The system of any of Clauses 175 through 179, wherein the one or more eye-tracking sensors and the one or more eye-tracking cameras are further configured to monitor fixation points of the user.

Clause 181. The system of any of Clauses 175 through 180, wherein the one or more eye-tracking sensors and the one or more eye-tracking cameras are further configured to monitor response times of the user.

Clause 182. The system of any of Clauses 175 through 181, wherein the VR headset further comprises speakers and vibrating motors, wherein the speakers and the vibrating motors are configured to provide auditory and haptic feedback to the user while the user completes the visual task and navigates the simulated underwater environment.

Clause 183. The system of any of Clauses 175 through 182, wherein the computing device is further configured to cause features of the simulated underwater environment to be changed.

Clause 184. The system of any of Clauses 175 through 183, wherein the computing device comprises an algorithm that is configured to process the user's eye movements to evaluate a user's visual clarity underwater.

Clause 185. The system of any of Clauses 175 through 184, wherein the computing device comprises an algorithm that is configured to process the user's eye movements to evaluate a user's depth perception underwater.

Clause 186. The system of any of Clauses 175 through 185, wherein the computing device comprises an algorithm that is configured to process the user's eye movements to evaluate a user's ability to adapt to changes in water conditions.

Clause 187. The system of any of Clauses 175 through 186, wherein the computing device comprises an algorithm that is configured to process the user's eye movements in real-time as the user completes the visual task.

Clause 188. The system of any of Clauses 175 through 187, wherein the computing device comprises an algorithm that is configured to adjust a difficulty of the visual task based on the user's eye movements.

Clause 189. The system of any of Clauses 175 through 188, wherein the computing device comprises an algorithm that is configured to adjust features of the simulated underwater environment based on the user's eye movements.

Clause 190. The system of any of Clauses 175 through 189, wherein the computing device is further configured to generate a report summarizing a user's performance of the underwater tasks and identifying areas for improvement.

Clause 191. A method for evaluating a user's cognitive workload in response to changing weather, the method comprising: displaying a virtual environment on screens of a virtual reality (VR) headset worn by a user; displaying a visual task in the virtual environment; displaying one or more weather features in the virtual environment; prompting the user to complete the visual task while the virtual environment includes the weather feature; monitoring eye movements, pupil dilation, heart rate, and brain wave patterns of the user as the user attempts the visual task; and processing the eye movements, the pupil dilation, the heart rate, and the brain wave patterns of the user to evaluate the user's cognitive workload.

Clause 192. The method of Clause 191, wherein displaying the virtual environment comprises displaying an outdoor environment.

Clause 193. The method of any of Clauses 191 through 192, wherein displaying the visual task in the virtual environment comprises prompting the user to navigate through environments with reduced visibility.

Clause 194. The method of any of Clauses 191 through 193, wherein displaying the visual task in the virtual environment comprises prompting the user to navigate through a maze.

Clause 195. The method of any of Clauses 191 through 194, wherein displaying the visual task in the virtual environment comprises prompting the user to navigate through an obstacle course.

Clause 196. The method of any of Clauses 191 through 195, wherein displaying the visual task in the virtual environment comprises prompting the user to identify objects positioned at different distances from the user.

Clause 197. The method of any of Clauses 191 through 196, wherein displaying the visual task in the virtual environment comprises prompting the user to locate objects positioned at different distances from the user.

Clause 198. The method of any of Clauses 191 through 197, wherein displaying the visual task in the virtual environment comprises prompting the user to collect objects positioned at different distances from the user.

Clause 199. The method of any of Clauses 191 through 198, wherein displaying the one or more weather features in the virtual environment comprises displaying the visual task in the virtual environment with bright sunlight.

Clause 200. The method of any of Clauses 191 through 199, wherein displaying the one or more weather features in the virtual environment comprises displaying the visual task in the virtual environment with fog.

Clause 201. The method of any of Clauses 191 through 200, wherein displaying the one or more weather features in the virtual environment comprises displaying the visual task in the virtual environment with rain.

Clause 202. The method of any of Clauses 191 through 201, wherein displaying the one or more weather features in the virtual environment comprises displaying the visual task in the virtual environment with snow.

Clause 203. The method of any of Clauses 191 through 202, wherein displaying the one or more weather features in the virtual environment comprises displaying the visual task in the virtual environment, the virtual environment being a low-light environment.

Clause 204. The method of any of Clauses 191 through 203, wherein displaying the one or more weather features in the virtual environment comprises displaying the visual task in the virtual environment with dynamically changing weather features.

Clause 205. The method of any of Clauses 191 through 204, further comprising, in response to processing the eye movements, the pupil dilation, the heart rate, and the brain wave patterns of the user, adjusting a difficulty and nature of the visual task.

Clause 206. The method of any of Clauses 191 through 205, further comprising, in response to processing the eye movements, the pupil dilation, the heart rate, and the brain wave patterns of the user, evaluating a visual clarity of the user.

Clause 207. The method of any of Clauses 191 through 206, further comprising, in response to processing the eye movements, the pupil dilation, the heart rate, and the brain wave patterns of the user, evaluating a reaction time of the user.

Clause 208. The method of any of Clauses 191 through 207, further comprising, in response to processing the eye movements, the pupil dilation, the heart rate, and the brain wave patterns of the user, evaluating a consistency of visual acuity of the user.

Clause 209. The method of any of Clauses 191 through 208, further comprising, in response to processing the eye movements, the pupil dilation, the heart rate, and the brain wave patterns of the user, evaluating a focusing consistency of the user.

Clause 210. The method of any of Clauses 191 through 209, further comprising providing auditory feedback to the user while the user completes the visual task.

Clause 211. The method of any of Clauses 191 through 210, further comprising providing haptic feedback to the user while the user completes the visual task.

Clause 212. The method of any of Clauses 191 through 211, further comprising generating a report summarizing a user's performance of the visual tasks and identifying areas for improvement.

Clause 213. A system for evaluating a user's cognitive workload in response to changing weather, the system comprising: a virtual reality (VR) headset worn by a user, the VR headset comprising: screens; one or more eye-tracking sensors and eye-tracking cameras, wherein the eye-tracking sensors and the eye-tracking cameras are configured to monitor eye movements, pupil dilation, and heart rate of the user; and one or more electrodes, wherein the electrodes are configured to monitor brain wave patterns of the user; and a computing device in electronic communication with the VR headset, the computing device being configured to cause a virtual environment with one or more weather features to be displayed on the screens of the VR headset, to cause a visual task to be displayed in the virtual environment, and to process the eye movements, the pupil dilation, the heart rate, and the brain wave patterns of the user to evaluate the user's cognitive workload.

Clause 214. The system of Clause 213, further comprising a handheld device in electronic communication with the VR headset and the computing device, wherein the handheld device is configured to receive input from the user as the user completes the visual task.

Clause 215. The system of any of Clauses 213 through 214, wherein the one or more eye-tracking sensors comprise ambient light sensors.

Clause 216. The system of any of Clauses 213 through 215, wherein the one or more eye-tracking cameras comprise infrared cameras.

Clause 217. The system of any of Clauses 213 through 216, wherein the one or more eye-tracking sensors and the one or more eye-tracking cameras are further configured to monitor fixation points of the user.

Clause 218. The system of any of Clauses 213 through 217, wherein the one or more eye-tracking sensors and the one or more eye-tracking cameras are further configured to monitor response times of the user.

Clause 219. The system of any of Clauses 213 through 218, wherein the one or more eye-tracking sensors and the one or more eye-tracking cameras are configured to monitor a gaze direction of the user.

Clause 220. The system of any of Clauses 213 through 219, wherein the one or more eye-tracking sensors and the one or more eye-tracking cameras are configured to monitor a fixation stability of the user.

Clause 221. The system of any of Clauses 213 through 220, wherein the one or more eye-tracking sensors and the one or more eye-tracking cameras are configured to monitor a visual response accuracy of the user.

Clause 222. The system of any of Clauses 213 through 221, wherein the VR headset further comprises speakers and vibrating motors, wherein the speakers and the vibrating motors are configured to provide auditory and haptic feedback to the user while the user completes the visual task and navigates the simulated underwater environment.

Clause 223. The system of any of Clauses 213 through 222, wherein the VR headset further comprises heat sensors configured to change a temperature experienced by the user in accordance with the one or more weather features of the virtual environment.

Clause 224. The system of any of Clauses 213 through 223, wherein the computing device comprises a software configured to dynamically adjust the virtual environment and the one or more weather features.

Clause 225. The system of any of Clauses 213 through 224, wherein the computing device comprises an algorithm configured to adjust the visual task based on how successfully the user completes the visual task.

Clause 226. The system of any of Clauses 213 through 225, wherein the computing device comprises an algorithm configured to adjust the visual task in real-time while the user completes the visual task based on how successfully the user completes the visual task.

Clause 227. The system of any of Clauses 213 through 226, wherein the computing device comprises an algorithm configured to process the eye movements, the pupil dilation, the heart rate, and the brain wave patterns of the user to evaluate a visual clarity of the user.

Clause 228. The system of any of Clauses 213 through 227, wherein the computing device comprises an algorithm configured to process the eye movements, the pupil dilation, the heart rate, and the brain wave patterns of the user to evaluate a reaction time of the user.

Clause 229. The system of any of Clauses 213 through 228, wherein the computing device comprises an algorithm configured to process the eye movements, the pupil dilation, the heart rate, and the brain wave patterns of the user to evaluate a consistency of visual acuity of the user.

Clause 230. The system of any of Clauses 213 through 229, wherein the computing device comprises an algorithm configured to process the eye movements, the pupil dilation, the heart rate, and the brain wave patterns of the user to evaluate a focusing consistency of the user.

Clause 231. The system of any of Clauses 213 through 230, wherein the computing device is further configured to generate a report summarizing a user's performance of the visual task and identifying areas for improvement.

Clause 232. A method for improving vision and reflexes, the method comprising: displaying a virtual environment on screens of a virtual reality (VR) headset worn by a user; displaying a first fast-moving object having a first speed and a first trajectory in the virtual environment; prompting the user to interact with the first fast-moving object; monitoring first eye movements of the user while the user interacts with the first fast-moving object; displaying a second fast-moving object having a second speed and a second trajectory in the virtual environment; prompting the user to interact with the second fast-moving object; monitoring second eye movements of the user while the user interacts with the second fast-moving object.

Clause 233. The method of Clause 232, wherein displaying the virtual environment comprises varying levels of brightness and contrast in the virtual environment.

Clause 234. The method of any of Clauses 232 through 233, wherein displaying the first and second fast-moving objects comprises displaying a ball.

Clause 235. The method of any of Clauses 232 through 234, wherein displaying the first and second fast-moving objects having the first and second speeds, respectively, comprises displaying the first and second fast-moving objects the first and second speeds being between approximately 30 mph and 90 mph.

Clause 236. The method of any of Clauses 232 through 235, wherein displaying the first and second fast-moving objects having the first and second speeds, respectively, comprises displaying the first and second fast-moving objects the first and second speeds being between approximately 50 mph and 100 mph.

Clause 237. The method of any of Clauses 232 through 236, wherein displaying the first and second fast-moving objects having the first and second speeds, respectively, comprises displaying the first and second fast-moving objects the first and second speeds being between approximately 70 mph and 130 mph.

Clause 238. The method of any of Clauses 232 through 237, wherein displaying the first and second fast-moving objects having the first and second speeds, respectively, comprises displaying the first and second fast-moving objects the first and second speeds being between approximately 100 mph and 350 mph.

Clause 239. The method of any of Clauses 232 through 238, wherein prompting the user to interact with the first and second fast-moving objects comprises prompting the user to run towards and catch the first and second fast-moving objects.

Clause 240. The method of any of Clauses 232 through 239, wherein prompting the user to interact with the first and second fast-moving objects comprises prompting the user to catch the first and second fast-moving objects.

Clause 241. The method of any of Clauses 232 through 240, wherein prompting the user to interact with the first and second fast-moving objects comprises prompting the user to predict the first and second trajectories.

Clause 242. The method of any of Clauses 232 through 241, wherein prompting the user to interact with the first and second fast-moving objects comprises prompting the user to track the first and second fast-moving objects with her eyes.

Clause 243. The method of any of Clauses 232 through 242, wherein prompting the user to interact with the first and second fast-moving objects comprises prompting the user to locate the first and second fast-moving objects with her eyes.

Clause 244. The method of any of Clauses 232 through 243, wherein prompting the user to interact with the first and second fast-moving objects comprises prompting the user to switch between focusing on the first fast-moving object and focusing on the second fast-moving object.

Clause 245. The method of any of Clauses 232 through 244, wherein prompting the user to interact with the first and second fast-moving objects comprises prompting the user to fuse the first and second fast-moving objects together with her eyes.

Clause 246. The method of any of Clauses 232 through 245, further comprising processing the first eye movements to determine the second speed and the second trajectory of the second fast-moving object.

Clause 247. The method of any of Clauses 232 through 246, further comprising processing the first eye movements in real-time while the user interacts with the first fast-moving object to determine the second speed and the second trajectory of the second fast-moving object.

Clause 248. The method of any of Clauses 232 through 247, further comprising providing auditory feedback to the user while the user interacts with the first and second fast-moving objects.

Clause 249. The method of any of Clauses 232 through 248, further comprising providing haptic feedback to the user while the user interacts with the first and second fast-moving objects.

Clause 250. The method of any of Clauses 232 through 249, further comprising generating a report summarizing a user's performance and identifying areas for improvement.

Clause 251. The method of any of Clauses 232 through 250, further comprising: simultaneously displaying the virtual environment on the screens of multiple VR headsets worn by multiple users; simultaneously displaying the first and second fast-moving objects for each user of the multiple users; monitoring the first and second eye movements of each user of the multiple users while each user interacts with the first and second fast-moving objects; and comparing the user's eye movements to the eye movements of each user of the multiple users.

Clause 252. The method of Clause 251, further comprising displaying each user of the multiple users in the virtual environment.

Clause 253. A system for improving vision and reflexes, the system comprising: a virtual reality (VR) headset worn by a user, the VR headset comprising screens, one or more eye-tracking sensors, and one or more eye-tracking cameras, the one or more eye-tracking sensors and the one or more eye-tracking cameras configured to monitor eye movements of the user; and a computing device in electronic communication with the VR headset, the computing device being configured to cause a virtual environment to be displayed on the screens, to cause a fast-moving object to be displayed in the virtual environment, and to process the eye movements of the user.

Clause 254. The system of Clause 253, further comprising a handheld device in electronic communication with the VR headset and the computing device, wherein the handheld device is configured to receive input from the user as the user interacts with the fast-moving object in the virtual environment.

Clause 255. The system of any of Clauses 253 through 254, wherein the one or more eye-tracking sensors comprise ambient light sensors.

Clause 256. The system of any of Clauses 253 through 255, wherein the one or more eye-tracking cameras comprise infrared cameras.

Clause 257. The system of any of Clauses 253 through 256, wherein the one or more eye-tracking sensors and the one or more eye-tracking cameras are further configured to monitor fixation points of the user.

Clause 258. The system of any of Clauses 253 through 257, wherein the one or more eye-tracking sensors and the one or more eye-tracking cameras are further configured to monitor response times of the user.

Clause 259. The system of any of Clauses 253 through 258, wherein the one or more eye-tracking sensors and the one or more eye-tracking cameras are further configured to monitor a gaze direction of the user.

Clause 260. The system of any of Clauses 253 through 259, wherein the one or more eye-tracking sensors and the one or more eye-tracking cameras are further configured to monitor a fixation stability of the user.

Clause 261. The system of any of Clauses 253 through 260, wherein the VR headset further comprises speakers and vibrating motors, wherein the speakers and the vibrating motors are configured to provide auditory and haptic feedback to the user while the user interacts with the fast-moving object.

Clause 262. The system of any of Clauses 253 through 261, wherein the computing device comprises an algorithm configured to process the eye movements of the user to evaluate a visual acuity of the user.

Clause 263. The system of any of Clauses 253 through 262, wherein the computing device comprises an algorithm configured to process the eye movements of the user to evaluate a depth perception of the user.

Clause 264. The system of any of Clauses 253 through 263, wherein the computing device comprises an algorithm configured to process the eye movements of the user to evaluate a peripheral awareness of the user.

Clause 265. The system of any of Clauses 253 through 264, wherein the computing device comprises an algorithm configured to process the eye movements of the user to evaluate a reaction speed of the user.

Clause 266. The system of any of Clauses 253 through 265, wherein the computing device comprises an algorithm configured to process the eye movements of the user in real-time as the user interacts with the fast-moving object.

Clause 267. The system of any of Clauses 253 through 266, wherein the computing device comprises an algorithm configured to process the eye movements of the user to adjust a speed and a trajectory of the fast-moving object.

Clause 268. The system of any of Clauses 253 through 267, wherein the computing device comprises an algorithm configured to process the eye movements of the user to adjust a brightness or a contrast of the virtual environment.

Clause 269. A method for evaluating vision safety protocols, the method comprising: displaying a virtual environment on screens of a virtual reality (VR) headset worn by a user; displaying visual hazards in the virtual environment; prompting the user to follow the vision safety protocols while completing a task in the virtual environment; monitoring a user's eye movements while the user completes the task; assessing a degree to which the user followed the vision safety protocols; and processing the user's eye movements to evaluate the vision safety protocols.

Clause 270. The method of Clause 269, wherein displaying the virtual environment comprises replicating a workplace.

Clause 271. The method of any of Clauses 269 through 270, wherein displaying the visual hazards comprises exposing the user to bright lights.

Clause 272. The method of any of Clauses 269 through 271, wherein displaying the visual hazards comprises displaying flying debris in the virtual environment.

Clause 273. The method of any of Clauses 269 through 272, wherein displaying the visual hazards comprises displaying high-speed machinery in the virtual environment.

Clause 274. The method of any of Clauses 269 through 273, wherein prompting the user to follow the vision safety protocols while completing the task comprises prompting the user to follow the vision safety protocols while navigating a vehicle through a construction site.

Clause 275. The method of any of Clauses 269 through 274, wherein prompting the user to follow the vision safety protocols while completing the task comprises prompting the user to follow the vision safety protocols while operating heavy machinery.

Clause 276. The method of any of Clauses 269 through 275, wherein prompting the user to follow the vision safety protocols while completing the task comprises prompting the user to follow the vision safety protocols while responding to healthcare emergencies.

Clause 277. The method of any of Clauses 269 through 276, wherein prompting the user to follow the vision safety protocols while completing the task comprises prompting the user to follow the vision safety protocols while using a tool to grab an object in the virtual environment.

Clause 278. The method of any of Clauses 269 through 277, wherein monitoring the user's eye movements comprises monitoring a user's gaze direction.

Clause 279. The method of any of Clauses 269 through 278, wherein monitoring the user's eye movements comprises monitoring a user's fixation stability.

Clause 280. The method of any of Clauses 269 through 279, wherein monitoring the user's eye movements comprises monitoring a user's reaction time.

Clause 281. The method of any of Clauses 269 through 280, further comprising processing the user's eye movements to assess the degree to which the user followed the vision safety protocols.

Clause 282. The method of any of Clauses 269 through 281, further comprising processing the user's eye movements in real-time while the user completes the task with the first fast-moving object to assess the degree to which the user followed the vision safety protocols.

Clause 283. The method of any of Clauses 269 through 282, further comprising providing auditory feedback to the user.

Clause 284. The method of any of Clauses 269 through 283, further comprising providing haptic feedback to the user.

Clause 285. The method of any of Clauses 269 through 284, further comprising: simultaneously displaying the virtual environment on the screens of multiple VR headsets worn by multiple users; simultaneously displaying the visual hazards in the virtual environment; prompting each user of the multiple users to follow the vision safety protocols while completing the task in the virtual environment; monitoring eye movements of each user of the multiple users while each user completes the task; assessing the degree to which each user of the multiple users followed the vision safety protocols; and processing the eye movements of each user of the multiple users to evaluate the vision safety protocols.

Clause 286. The method of Clause 285, further comprising displaying each user of the multiple users in the virtual environment.

Clause 287. The method of any of Clauses 269 through 286, further comprising adjusting the vision safety protocols and prompting the user to follow the adjusted vision safety protocols while completing the task in the virtual environment.

Clause 288. The method of any of Clauses 269 through 287, further comprising generating a report summarizing an effectiveness of the vision safety protocols and recommending improvements.

Clause 289. A system for evaluating vision safety protocols, the system comprising: a virtual reality (VR) headset worn by a user, the VR headset comprising screens, one or more eye-tracking sensors, and one or more eye-tracking cameras, the one or more eye-tracking sensors and the one or more eye-tracking cameras configured to monitor eye movements of the user and a degree to which the user follows the vision safety protocols while completing a task in a virtual environment; and a computing device in electronic communication with the VR headset, the computing device being configured to cause the virtual environment with visual hazards to be displayed on the screens and to process the eye movements of the user.

Clause 290. The system of Clause 289, further comprising a handheld device in electronic communication with the VR headset and the computing device, wherein the handheld device is configured to receive input from the user as the user interacts with the fast-moving object in the virtual environment.

Clause 291. The system of any of Clauses 289 through 290, further comprising one or more motion-tracking sensors configured to track physical movements and a spatial orientation of the user, wherein the computing device is further configured to process the physical movements and the spatial orientation of the user.

Clause 292. The system of any of Clauses 289 through 291, wherein the one or more eye-tracking sensors comprise ambient light sensors.

Clause 293. The system of any of Clauses 289 through 292, wherein the one or more eye-tracking cameras comprise infrared cameras.

Clause 294. The system of any of Clauses 289 through 293, wherein the one or more eye-tracking sensors and the one or more eye-tracking cameras are configured to monitor a gaze direction of the user.

Clause 295. The system of any of Clauses 289 through 294, wherein the one or more eye-tracking sensors and the one or more eye-tracking cameras are configured to monitor a fixation stability of the user.

Clause 296. The system of any of Clauses 289 through 295, wherein the one or more eye-tracking sensors and the one or more eye-tracking cameras are configured to monitor a reaction time of the user.

Clause 297. The system of any of Clauses 289 through 296, wherein the one or more eye-tracking sensors and the one or more eye-tracking cameras are configured to monitor saccadic movements of the user.

Clause 298. The system of any of Clauses 289 through 297, wherein the one or more eye-tracking sensors and the one or more eye-tracking cameras are configured to monitor smooth pursuit movements of the user.

Clause 299. The system of any of Clauses 289 through 298, wherein the computing device comprises an algorithm configured to evaluate the vision safety protocols by processing the degree to which the user follows the vision safety protocols.

Clause 300. The system of any of Clauses 289 through 299, wherein the computing device comprises an algorithm configured to adjust the vision safety protocols upon processing the degree to which the user follows the vision safety protocols.

Clause 301. The system of any of Clauses 289 through 300, wherein the computing device is further configured to generate a report summarizing an effectiveness of the vision safety protocols and recommending improvements, wherein the computing device comprises a user interface at which the report can be accessed.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim can include some or all of the words (e.g., steps, operations, means or components) recited in a clause, a sentence, a phrase or a paragraph. In one aspect, a claim can include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components can include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that can include at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “can include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies, and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,” such as “comprise” and “can comprise,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) can include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology can include other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method for evaluating a user's underwater vision, the method comprising:
displaying a simulated underwater environment on a screen of a VR headset worn by a user;
administering a visual task in the simulated underwater environment;
monitoring a user's eye movements while the user completes the visual task; and
comparing the user's eye movements to a database to evaluate the user's underwater vision.

2. The method of claim 1, wherein administering the visual task comprises prompting the user to identify objects in the simulated underwater environment, wherein the objects are positioned at different distances away from the user and at different depths of the simulated underwater environment.

3. The method of claim 1, wherein administering the visual task comprises prompting the user to grab objects in the simulated underwater environment, wherein the objects are positioned at different distances away from the user and at different depths of the simulated underwater environment.

4. The method of claim 1, wherein administering the visual task comprises prompting the user to use a tool to collect objects in the simulated underwater environment, wherein the objects are positioned at different distances away from the user and at different depths of the simulated underwater environment.

5. The method of claim 1, wherein administering the visual task comprises prompting the user to navigate around obstacles in the simulated underwater environment.

6. The method of claim 1, wherein administering the visual task comprises prompting the user to navigate through different lighting conditions in the simulated underwater environment.

7. The method of claim 1, wherein administering the visual task comprises prompting the user to follow a moving object through the simulated underwater environment.

8. The method of claim 1, wherein monitoring the user's eye movements comprises monitoring a user's gaze direction.

9. The method of claim 1, wherein monitoring the user's eye movements comprises monitoring a user's fixation stability.

10. The method of claim 1, wherein monitoring the user's eye movements comprises monitoring a user's response accuracy.

11. The method of claim 1, wherein monitoring the user's eye movements comprises monitoring a user's saccadic movements.

12. The method of claim 1, wherein monitoring the user's eye movements comprises monitoring a user's smooth pursuit movements.

13. The method of claim 1, further comprising processing the user's eye movements in real-time as the user completes the visual task to adjust a difficulty of the visual task while the user is completing the visual task.

14. The method of claim 13, wherein processing the user's eye movements comprises evaluating a user's visual clarity underwater.

15. The method of claim 13, wherein processing the user's eye movements comprises evaluating a user's depth perception underwater.

16. The method of claim 13, wherein processing the user's eye movements comprises evaluating a user's ability to adapt to changes in water conditions.

17. The method of claim 1, further comprising:
simultaneously displaying the simulated underwater environment on the screen of multiple VR headsets worn by multiple users;
simultaneously administering the visual task in the simulated underwater environment for each user of the multiple users;
monitoring the eye movements of each user of the multiple users while each user completes the visual task; and
comparing the user's eye movements to the eye movements of each user of the multiple users.

18. The method of claim 17, further comprising displaying each user of the multiple users in the simulated underwater environment.

19. The method of claim 17, further comprising adjusting features of the simulated underwater environment displayed on the screen of each VR headset of the multiple VR headsets.

20. A system for testing vision in simulated underwater environments, the method comprising:
a virtual reality (VR) headset worn by a user, the VR headset comprising a screen, one or more eye-tracking sensors, and one or more eye-tracking cameras, the one or more eye-tracking sensors and the one or more eye-tracking cameras configured to monitor eye movements, fixation patterns, and blink rates of the user; and
a computing device in electronic communication with the VR headset, the computing device being configured to cause a simulated underwater environment to be displayed on the screen, to cause a visual task to be displayed in the simulated underwater environment, and to process the eye movements, the fixation patterns, and the blink rates of the user.
